(19) United States
(12) Patent Application Publication
(10) Pub. No.: US 2003/0170252 A1
(43) Pub. Date: Sep. 11, 2003

(54) Antibody compositions specific for p33QIK and p63Krs1 polypeptides and uses thereof

(76) Inventor: Hwa-Chain Robert Wang, Knoxville, TN (US)

Correspondence Address: WILLIAMS, MORGAN & AMERSON, P.C., 10333 RICHMOND, SUITE 1100, HOUSTON, TX, 77042, US

(21) Appl. No.: 09/822,110

(22) Filed: Mar. 30, 2001

Related U.S. Application Data
(60) Provisional application No. 60/193,550, filed on Mar. 31, 2000.

(51) Int. Cl. A61K 39/00; C07K 5/00; C07K 7/00; C07K 16/00; C07K 17/00; A61K 38/00; C12N 5/06; C12N 5/16; C07K 1/00; C07K 14/00; C12P 21/08; C12N 1/00

(52) U.S. Cl. 424/185.1; 530/324; 530/350; 530/387.3; 435/326; 435/810

(57) ABSTRACT

Disclosed are compositions and methods for the detection and quantitation of mammalian p33^{QIK} and p63^{Krsl} polypeptides in a sample. Also disclosed are methods, kits, and reagents for the diagnosis of diseases manifesting in or resulting from altered p33^{QIK} and/or p63^{Krsl} polypeptide levels in a patient.

### 1.0 BACKGROUND OF THE INVENTION

The present application claims priority to U.S. Provisional Patent Application Serial No. 60/193,550, filed Mar. 31, 2000; the entire specification, claims and figures of which are incorporated herein by reference without disclaimer. The United States government has certain rights to the present invention pursuant to Grant Number 1R29 CA69530 from the National Cancer Institute.

### 1.1 FIELD OF THE INVENTION

The present invention relates to immunological compositions specific for mammalian p33^{QIK} and p63^{Krsl} peptides and polypeptides, and methods of making and using p33^{QIK}- and p63^{Krsl}-specific antibodies, and antigen binding fragments thereof in a variety of detection, diagnostic and therapeutic regimens. The invention provides new and effective methods, compositions and kits for eliciting immune and T cell responses to p33^{QIK} and p63^{Krsl} peptides, polypeptides, and antigenic fragments thereof in a mammal.

### 1.2 DESCRIPTION OF RELATED ART

The control of a shift between G0 and G1 phases is believed to be the main determinant of cell proliferation rate and cell differentiation. Many types of cells can remain healthy in a nonproliferative quiescent state in vivo for a long time (Denhardt et aL, 1986; Cross et al, 1989). Normal cells in cultures that lack growth factors become growth-arrested in quiescent state (Campisi et al., 1984; Larsson et al., 1985). Failure to control the shift from G0 to G1 phase, with resulting cell proliferation, is also believed to be the main defective factor in many cancer cells, contributing to the decreased dependence of transformed cells on growth factors for cell growth (Pardee, 1989).

Several growth-arrest-specific (gas) genes have been reported, based on their preferential expression in quiescent cultures and down-regulation upon entry of cells into the cell cycle (Schneider et al., 1988; Ciccarelli et al., 1990; Manfioletti et al., 1990; 1993; Welcher et al., 1991; Coccia et al., 1992). Although the roles of these gene products in cell quiescence are unclear, it is reasonable to believe that a particular signaling pathway is required to control cell machinery in quiescent state.

During the G0/G1 transition re-entry of cells into the cell cycle requires activation of cell growth signaling pathways. The mitogen-activated signaling pathway, which comprises Ras, Raf, Mek, Erk, and Rsk, plays an important role in induction of transcription factors and in expression of the immediate early G1 gene products, leading to induction of secondary cellular events to complete the cell cycle (Gille et al., 1992; Blenis, 1993; Whitmarsh et al., 1995).

The Krsl gene encodes a protein kinase, p63^{Krsl}, of which a proteolytic product, p33/36 is thought to be activated in cancer cells following treatment with anticancer agents. Thus detection of p33/36 activity has been recognized as useful as a therapeutic efficacy marker in anticancer clinical studies using different anticancer drugs. However, there have been no reports of antibodies that selectively recognize the native form of p33/36 polypeptides.

One known commercially available peptide antibody is Krsl(N19) (Santa Cruz Biotechnology, Santa Cruz, Calif.) which was raised against a peptide corresponding to an amino-terminal region of the Krsl peptide. It also reacts with Krsl and p36 in western immunoblotting. However, based on recent studies, this specific antibody was not capable of immunoprecipitating either Krsl or p33/36 from cell lysates, indicating that this antibody did not recognize the native form of Krsl and p33/36. Therefore, the usage of this antibody is limited to recognize the denatured form of Krsl. Since this antibody cannot recognize the native form of Krsl and p33/36, it cannot be used to isolate these kinases from cell or tissue samples thus limiting the use of this antibody to detection of the denatured forms of the Krsl and p33/36 polypeptides.

### 2.0 SUMMARY OF THE INVENTION

The present invention overcomes the limitations inherent in the prior art by providing compositions and methods for detecting and quantatiting mammalian p33^{QIK} or p63^{Krsl} peptides or polypeptides, and in particular antibody and antigen binding fragments thereof that specifically detect and quantitate the presence of p33^{QIK} and p63^{Krsl} peptides and polypeptides in their native, biologically-active forms. The invention also provides a method of generating an immune or a T cell response in an animal, and in particular in a mammal such as a human. These methods concern in a general sense the administration of at least a first composition to the animal that comprises at least a first isolated peptide of from 9 to about 80 amino acids in length, or at least a first nucleic acid segment that encodes such a peptide, wherein the peptide comprises, consists essentially of, or consists of a first contiguous amino acid sequence according to any one of SEQ ID NO:3 through SEQ ID NO:79. Particularly preferred compositions include those peptides that comprise, consist essentially of, or consist of, the amino acid sequence of SEQ ID NO:3 or SEQ ID NO:4.

Methods and compositions are also provided for diagnosis, detection, treatment, and prognostication of p33^{QIK} or p63^{Krsl}-mediated diseases, and the detection and quantitation of p33^{QIK} and p63^{Krsl} peptides and antibodies in a sample, and in particular, in biological samples including clinical samples that may be obtained from a human patient. Also disclosed herein are antibody-producing hybridoma cell lines, polyclonal sera, pluralities of antibodies and antigen binding fragments produced therefrom, and methods for their use in detecting native or biologically-active forms of the mammalian p33^{QIK} or p63^{Krsl} polypeptide in a sample. Methods and compositions are also provided for inhibiting the activity of p33^{QIK} or p63^{Krsl} polypeptides in a cell, and for diagnosing and ameliorating cellular abnormalities resulting from the expression or overexpression of one or more p33^{QIK} or p63^{Krsl} polypeptides.

The importance of developing multi-functional antibodies that were immunospecific for p33^{QIK} and/or p63^{Krsl} polypeptides, and in particular, biologically active forms of these polypeptides, for use in a variety of diagnostic and therapeutic regimens was keenly recognized by the inventor, and represents a significant advance in the field. In one illustrative embodiment, the invention provides an exemplary antiserum, designated Krsl-NC, that was raised against a peptide corresponding to the sixteen contiguous amino acid residues from position 296 to position 311 (SEQ ID NO:3) of the p63^{Krsl} polypeptide in rabbits. This Krsl-NC antibody is able to immunoprecipitate native, or biologically-active forms of both the p63^{Krsl} and the p33^{QIK} polypeptides described herein, and as such, offers an important advance in the field by providing for the first time immunologic compositions that are specific for the native form of these polypeptides. This antibody, and other antibodies like is has demonstrated its utility in determining the protein level and kinase activity of p63^{Krsl} and p33^{QIK} peptides and polypeptides in a variety of diagnostic assays, such as western immunoblotting, immunoprecipitation followed by kinase assay, and immunohistochemistry. In one aspect of the invention, this antibody, and antibodies that recognize the same or substantially the same epitope(s) as this antibody, may be employed in methods for the detection of biologically-active forms of p63^{Krsl} and p33^{QIK} peptides and polypeptides, such as, for example, in methods for assessing drug efficacy and molecular mechanisms in anticancer therapeutics, and in methods of monitoring diseases and biological pathways that are affected by the activity of p63^{Krsl} and p33QIK polypeptides. The antibodies of the invention that are immunospecific for the native, non-denatured forms of the p63^{Krsl} and p33^{QIK} polypeptides may also be employed in one or more therapeutic regimens as described herein, and particularly in those methods that inhibit or alter the biological activity of one or more such polypeptides in vitro or in vivo. Preferably, the p63^{Krsl} and p33^{QIK} -specific antibodies and antigen binding fragments of the invention interact with their target polypeptides in such a manner that the activity of the polypeptides is altered, modulated, reduced, diminished, decreased, or inhibited. In an illustrative embodiment, such antibody compositions as disclosed herein may be used to alter, reduce, or inhibit the activity of a p63^{Krsl} or p33^{QIK} polypeptide in a cell. In one such method, an antibody composition of the invention is provided to such a cell in an amount and for a time sufficient to alter, reduce, or inhibit the activity of the polypeptide. The cell may be contacted with the antibody composition in vitro, such as in a cell assay or an ex situ

In another exemplary embodiment, one aspect of the present invention concerns pluralities of antibodies obtained from a polyclonal antiserum that are also immunospecific for one or more antigenic epitopes on the native Krsl polypeptide. This antiserum was prepared using a second internal 14-amino acid antigenic peptide fragment corresponding to amino acid residues 312 to 325 (SEQ ID NO:4) of the native Krsl polypeptide. This epitope was purified and used to immunize rabbits and produce the polyclonal antiserum. The titer of antibodies to this second antigenic epitopic Krsl-derived peptide was as high as 10 K in a standard ELISA. Although this antiserum did not interact with p63^{Krsl} or p33^{QIK} polypeptides in immunoprecipitation and western immunoblotting assays as well as the Krsl-NC antibody described above, it was nevertheless useful in specifically immunoprecipitating the native forms of the p63^{Krsl} and p33^{QIK} polypeptides.

From these results, the inventor determined that particularly preferred antigenic peptides could be identified within the primary amino acid sequence of the native Krsl polypeptide, and such peptides could be selected for their ability to elicit an immune response in an animal and to give rise to a polyclonal antiserum from which specific antibodies could be identified which were able to immunoprecipitate both the p63^{Krsl} and p33^{QIK} polypeptides in their native forms. Exemplary antigenic epitopes useful in the generation of antibodies that are immunospecific for portions of the native Krsl polypeptide have been shown in SEQ ID NO:3 through SEQ ID NO:79. The sequence of these epitopic peptides isolated from the native Krsl polypeptide (SEQ ID NO:2) are also shown in Example 2. For convenience, the DNA sequence encoding native Krsl polypeptide is also shown in SEQ ID NO:1.

Antibodies immunospecific for one or more of the selected epitopic regions of p63^{Krsl} disclosed in SEQ ID NO:3 through SEQ ID NO:79, are also part of the invention. In one embodiment, there is provided a monoclonal antibody that binds immunologically to a mammalian p33^{QIK} or p63^{Krsl} polypeptide. The preferred antibodies of the invention may be non-cross reactive with other polypeptides, including human polypeptides, or they may bind to non-human p33^{QIK} or p63^{Krsl} polypeptides, but not to human p33^{QIK} or p63^{Krsl} polypeptides. Certain of the antibodies of the invention are preferably immunoreactive with native, or biologically-active forms of the p33^{QIK} or p63^{Krsl} polypeptides, but are preferably marginally- or non-immunoreactive with denatured or inactive forms of the p33^{QIK} or p63^{Krsl} polypeptides including for example, polypeptides denatured by gel electrophoresis methods employing agents such as sodium dodecyl sulfate (SDS).

Also encompassed are hybridoma cells and cell lines producing such antibodies, polyclonal antiserum, and antibodies purified from such a polyclonal antiserum which bind immunologically to biologically-active mammalian p33^{QIK} or p63^{Krsl} polypeptides. The antisera may be derived from any animal, but preferably is from an animal other than a human. Preferred antigens for the preparation of such sera include a p33^{QIK} or p63^{Krsl} polypeptide isolated from a human, rat, goat, rabbit, pig, horse, cat, dog, hamster, monkey or other such animal. Preferred hosts for the preparation of polyclonal antisera specific for a mammalian p33^{QIK} or p63^{Krsl} polypeptide include animals such as rabbits, goats, and other such animals. An exemplary polyclonal serum is the antiserum designated Ab-KQ as described herein.

The invention also provides pharmaceutical compositions that comprise one or more of the mammalian p33^{QIK} or p63^{Krsl}-specific compositions disclosed herein. Such compositions may include mammalian p33^{QIK} or p63^{Krsl} polypeptide preparations themselves, or mammalian p33^{QIK} or p63^{Krsl} antibodies, antisera, antigens, peptide epitopes, protein fusions, peptides and the like.

The invention further provides diagnostic kits for use in the detection of human p33^{QIK} or p63^{Krsl} polypeptides and in the diagnosis of p33^{QIK} or p63^{Krsl}-mediated disorders. Such kits preferably comprise one or more p33^{QIK} or p63^{Krsl}-specific antibody compositions as disclosed herein, and instructions for their use in diagnostic or therapeutic tests.

In another embodiment, the invention also concerns various methods of detecting p33^{QIK} or p63^{Krsl} polypeptides in a sample. The method generally involves contacting a sample suspected of containing one or more p33^{QIK} or p63^{Krsl} polypeptides with an antibody composition as disclosed herein, under conditions effective to produce immune complexes and detecting the complexes so formed. The sample is preferably a clinical sample, and may be a blood or serum sample obtained from a human patient.

In still yet another embodiment there is provided a method of diagnosing p33^{QIK} or p63^{Krsl}-mediated disease. The method generally involves obtaining a biological sample from a subject; and quantitating the level of p33^{QIK} or p63^{Krsl} polypeptide in the sample. The sample is a cell, cell culture, tissue or fluid sample, and may be of clinical or non-clinical origin. Preferably, it is a blood sample.

2.1 Screening Methods and Immunodetection Kits

The present invention also provides compositions, methods and kits for screening samples suspected of containing mammalian p33^{QIK} or p63^{Krsl} polypeptides. Such samples are preferably biological samples, and as such, the sample can be any tissue or fluid. Fluid samples such as peripheral blood, serum, lymph fluid, ascites, serous fluid, or the like may also be analyzed for p33^{QIK} or p63^{Krsl}-specific antigens.

Such screening may be performed on samples such as transformed host cells, clinical or laboratory samples suspected of containing or producing such polypeptides. The kit can contain reagents for detecting an interaction between a sample and an antibody of the present invention. The provided reagent can be radio-, fluorescently- or enzymatically-labeled. The kit can contain a known radiolabeled agent capable of binding or interacting with a nucleic acid, polypeptide, or antibody of the present invention.

The reagent of the kit can be provided as a liquid solution, attached to a solid support or as a dried powder. Preferably, when the reagent is provided in a liquid solution, the liquid solution is an aqueous solution. Preferably, when the reagent provided is attached to a solid support, the solid support can be chromatograph media, a test plate having a plurality of wells, or a microscope slide. When the reagent provided is a dry powder, the powder can be reconstituted by the addition of a suitable solvent that may be provided.

In still further embodiments, the present invention concerns immunodetection methods and associated kits. It is proposed that the p33^{QIK} or p63^{Krsl} polypeptides of the present invention may be employed to detect antibodies having reactivity therewith, or, alternatively, antibodies prepared in accordance with the present invention, may be employed to detect p33^{QIK} or p63^{Krsl} polypeptides in a sample. In general, these methods will include first obtaining a sample suspected of containing such polypeptides, contacting the sample with an antibody in accordance with the present invention, as the case may be, under conditions effective to allow the formation of an immunocomplex, and then detecting the presence of the immunocomplex. Alternatively, when a sample is suspected of containing an antibody specific for such p33^{QIK} or p63^{Krsl} polypeptides, these methods will involve contacting the sample with a mammalian p33^{QIK} or p63^{Krsl} polypeptide in accordance with the present invention, as the case may be, under conditions effective to allow the formation of an immunocomplex, and then detecting the presence of the immunocomplex.

In general, the detection of immunocomplex formation is quite well known in the art and may be achieved through the application of numerous approaches. For example, the present invention contemplates the application of ELISA, RIA, immunoblot (e.g., dot blot), indirect immunofluorescence techniques and the like. Generally, immunocomplex formation will be detected through the use of a label, such as a radiolabel or an enzyme tag (such as alkaline phosphatase, horseradish peroxidase, or the like). Of course, one may find additional advantages through the use of a secondary binding ligand such as a second antibody or a biotin/avidin ligand binding arrangement, as is known in the art.

For assaying purposes, it is proposed that virtually any sample suspected of comprising either a p33^{QIK} or p63^{Krsl} polypeptide, or a p33^{QIK} or p63^{Krsl}-specific antibody sought to be detected, as the case may be, may be employed. It is contemplated that such embodiments may have application in the titering of antigen or antibody samples, in the selection of hybridomas, and the like. In related embodiments, the present invention contemplates the preparation of kits that may be employed to detect the presence of p33^{QIK} or p63^{Krsl}-specific antibodies in a sample. Samples may include cells, cell supernatants, cell suspensions, cell extracts, enzyme fractions, protein extracts, or other cell-free compositions suspected of containing such compositions. Generally speaking, kits in accordance with the present invention will include a suitable p33^{QIK} or p63^{Krsl} polypeptide, or an antibodythat is specific for a p33^{QIK} or p63^{Krsl} polypeptide, together with an immunodetection reagent and a means for containing the antibody or antigen and reagent. The immunodetection reagent will typically comprise a label associated with the antibody or antigen, or associated with a secondary binding ligand. Exemplary ligands might include a secondary antibody directed against the first antibody or antigen or a biotin or avidin (or streptavidin) ligand having an associated label. Of course, as noted above, a number of exemplary labels are known in the art and all such labels may be employed in connection with the present invention.

The container will generally include a vial into which the antibody, antigen or detection reagent may be placed, and preferably suitably aliquotted. The kits of the present invention will also typically include a means for containing the antibody, antigen, and reagent containers in close confinement for commercial sale. Such containers may include injection or blow-molded plastic containers into which the desired vials are retained.

2.2 Antigenic Peptide and Polypeptide Compositions

The present invention also provides for the use of mammalian p33^{QIK} or p63^{Krsl} polypeptides and peptides and epitopes derived therefrom for the immunization of animals relating to the production of antibodies. It is envisioned that in certain embodiments, p33^{QIK} or p63^{Krsl} polypeptides, or fragments thereof, may be prepared, denatured, coupled, bonded, bound, conjugated or chemically-linked to one or more agents via linkers, polylinkers or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyannin (KLH) or bovine serum albumin (BSA).

In one embodiment, the invention provides a method of generating an immune or a T cell response in an animal, and in particular in a mammal such as a human. The method concerns in a general sense the administration of at least a first composition to the animal that comprises at least a first isolated peptide of from 9 to about 80 amino acids in length, or at least a first nucleic acid segment that encodes such a peptide, wherein the peptide comprises a first contiguous amino acid sequence according to any one of SEQ ID NO:3 to SEQ ID NO:76, and more particularly, a contiguous amino acid sequence according to any one of SEQ ID NO:3 through SEQ ID NO:46, with peptides comprising one or more of the primary amino acid sequences disclosed in SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20 being particularly preferred.

The invention encompasses peptides that may be of any intermediate length in the preferred ranges, such as for example, those peptides of about 75, about 70, about 65, about 50, about 55, about 50, about 45, about 40, about 35, about 30, about 25, about 20, or even about 15 amino acids or so in length, as well as those peptides having intermediate lengths including all integers within these ranges (e.g., the peptides may be about 79, about 78, about 77, about 76, about 74, about 73, about 72, about 71, about 69, about 68, about 67, about 66, about 64, about 63, about 62, or even about 61 or so amino acids in length, or alternatively, the peptides may be about 59, about 58, about 57, about 56, about 54, about 53, about 52, about 51, about 49, about 48, about 47, about 46, about 44, about 43, about 42, about 41, about 39, about 38, about 27, or even about 36 or so amino acids in length, etc.). In particular embodiments, when smaller peptides are preferred, the length of the peptide may be 9, or about 10, about 11, about 12, about 13, about 14, about 15, about 16, about 17, about 18, about 19, or even about 20 or so amino acids in length, so long as the peptide comprises at least a first contiguous amino acid sequence according to any one of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, as well as any one of SEQ ID NO:21 to SEQ ID NO:30, SEQ ID NO:31 to SEQ ID NO:40, SEQ ID NO:41 to SEQ ID NO:50, SEQ ID NO:51 to SEQ ID NO:60, SEQ ID NO:61 to SEQ ID NO:70, or SEQ ID NO:71 to SEQ ID NO:76. Likewise, when slightly longer peptides are preferred, the length of the peptide may be about 21, or about 22, or about 23, or about 24, or even about 25 or so amino acids in length, so long as the peptide comprises at least a first contiguous amino acid sequence according to any one of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, as well as any one of SEQ ID NO:21 to SEQ ID NO:30, SEQ ID NO:31 to SEQ ID NO:40, SEQ ID NO:41 to SEQ ID NO:50, SEQ ID NO:51 to SEQ ID NO:60, SEQ ID NO:61 to SEQ ID NO:70, or SEQ ID NO:71 to SEQ ID NO:76. When intermediate-length antigenic peptides or antigen binding fragments are desired, the peptides may be on the order of about 26, or about 27, or about 28, or about 29, or about 30, or about 31, or about 32, or about 33, or about 34, or even about 35 or so amino acids in length, so long as they each comprise at least a first contiguous amino acid sequence according to any one of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, as well as any one of SEQ ID NO:21 to SEQ ID NO:30, SEQ ID NO:31 to SEQ ID NO:40, SEQ ID NO:41 to SEQ ID NO:50, SEQ ID NO:51 to SEQ ID NO:60, SEQ ID NO:61 to SEQ ID NO:70, or SEQ ID NO:71 to SEQ ID NO:76.

These peptides comprise at least a first contiguous amino acid sequence according to any one of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, as well as any one of SEQ ID NO:21 to SEQ ID NO:30, SEQ ID NO:31 to SEQ ID NO:40, SEQ ID NO:41 to SEQ ID NO:50, SEQ ID NO:51 to SEQ ID NO:60, SEQ ID NO:61 to SEQ ID NO:70, or SEQ ID NO:71 to SEQ ID NO:76, but may also, optionally comprise at least a second, at least a third, or even at least a fourth or greater contiguous amino acid sequence according to any one of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, as well as any one of SEQ ID NO:21 to SEQ ID NO:30, SEQ ID NO:31 to SEQ ID NO:40, SEQ ID NO:41 to SEQ ID NO:50, SEQ ID NO:51 to SEQ ID NO:60, SEQ ID NO:61 to SEQ ID NO:70, or SEQ ID NO:71 to SEQ ID NO:76. A single peptide may contain only one of the contiguous amino acid sequences disclosed herein, or alternatively, a single peptide may comprise a plurality of contiguous amino acid sequences according to any one of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, as well as any one of SEQ ID NO:21 to SEQ ID NO:30, SEQ ID NO:31 to SEQ ID NO:40, SEQ ID NO:41 to SEQ ID NO:50, SEQ ID NO:51 to SEQ ID NO:60, SEQ ID NO:61 to SEQ ID NO:70, or SEQ ID NO:71 to SEQ ID NO:76. In fact, the peptide may comprise a plurality of the same contiguous amino acid sequences, or they may comprise one or more different contiguous amino acid sequences disclosed in SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, as well as any one of SEQ ID NO:21 to SEQ ID NO:30, SEQ ID NO:31 to SEQ ID NO:40, SEQ ID NO:41 to SEQ ID NO:50, SEQ ID NO:51 to SEQ ID NO:60, SEQ ID NO:61 to SEQ ID NO:70, or SEQ ID NO:71 to SEQ ID NO:76. For example, a single peptide of from 9 to about 50 amino acids in length could comprise a single epitopic peptide disclosed herein, or could comprise 2, 3, 4, or even 5 distinct epitopic sequences as disclosed in any of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, as well as any one of SEQ ID NO:21 to SEQ ID NO:30, SEQ ID NO:31 to SEQ ID NO:40, SEQ ID NO:41 to SEQ ID NO:50, SEQ ID NO:51 to SEQ ID NO:60, SEQ ID NO:61 to SEQ ID NO:70, or SEQ ID NO:71 to SEQ ID NO:76. Alternatively, a single peptide of from 9 to about 50 amino acids in length could comprise 2, 3, 4, or even 5 identical epitopic sequences as disclosed in any one of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, as well as any one of SEQ ID NO:21 to SEQ ID NO:30, SEQ ID NO:31 to SEQ ID NO:40, SEQ ID NO:41 to SEQ ID NO:50, SEQ ID NO:51 to SEQ ID NO:60, SEQ ID NO:61 to SEQ ID NO:70, or SEQ ID NO:71 to SEQ ID NO:76.

In one exemplary embodiment, the peptide composition comprises at least a first isolated peptide of from 9 to about 80 amino acids in length, or at least a first nucleic acid segment that encodes such a peptide; wherein the peptide comprises at least a first contiguous amino acid sequence selected from the group consisting of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, as well as any one of SEQ ID NO:21 to SEQ ID NO:30, SEQ ID NO:31 to SEQ ID NO:40, SEQ ID NO:41 to SEQ ID NO:50, SEQ ID NO:51 to SEQ ID NO:60, SEQ ID NO:61 to SEQ ID NO:70, or SEQ ID NO:71 to SEQ ID NO:76.

Preferred peptides of the present invention likewise encompass those from 10 to about 70 amino acids in length, those from 11 to about 60 amino acids in length, those from 12 to about 60 amino acids in length, those from 13 to about 60 amino acids in length, as well as those from 14 to about 70 amino acids in length, and those from 15 to about 70 amino acids in length. Likewise, preferred peptides of the present invention encompass those from 16 to about 70 amino acids in length, and any and all lengths, and sub-ranges of lengths within the overall preferred range of peptides of from 9 to about 70 amino acids or so in length. In similar fashion, the invention also encompasses those peptides having a length of from 10 or 11 to about 55 or 70 amino acids in length, and those having a length of from 12 or 13 to about 45 or 50 amino acids in length, as well as those peptides having a length of from 14 or 15 to about 35 or 40 amino acids in length, those peptides having a length of from 16 or 17 to about 25 or 30 amino acids in length, and those peptides having a length of from 18 or 19 to about 20 or so amino acids in length, and so on, to include all sub-ranges within the overall range of from 9 to about 60 amino acids in length.

Throughout this disclosure, a phrase such as “a sequence as disclosed in SEQ ID NO:21 to SEQ ID NO:30” is intended to encompass any and all contiguous amino acid sequences disclosed by any of these sequence identifiers, and particularly, the peptide sequences disclosed in Example 2 of the present specification. That is to say, “a sequence as disclosed in any of SEQ ID NO:21 through SEQ ID NO:30” means a sequence that is disclosed in SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29 or SEQ ID NO:30. In fact, the invention encompasses peptides and polynucleotides encoding them that comprise at least a first contiguous amino acid sequence as disclosed in any one of the sequences identified as SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, as well as any one of SEQ ID NO:21 to SEQ ID NO:30, SEQ ID NO:31 to SEQ ID NO:40, SEQ ID NO:41 to SEQ ID NO:50, SEQ ID NO:51 to SEQ ID NO:60, SEQ ID NO:61 to SEQ ID NO:70, or SEQ ID NO:71 to SEQ ID NO:76.

The polypeptides of the invention also encompass those polypeptides that comprise a biologically-active p33^{QIK} molecule, and preferably those polypeptides that consist essentially of, or consist of, an amino acid sequence of from about position 1, 2 or 3 of SEQ ID NO:2 to about position 319, 320, 321, or 322 of SEQ ID NO:2. As such, polypeptides that consist essentially of, or consist of, an amino acid sequence of from position 1 of SEQ ID NO:2 to about position 322 of SEQ ID NO:2, and those peptides that consist essentially of, or consist of, an amino acid sequence of from about amino acid residue 1 to about amino acid residue 321 of SEQ ID NO:2 are considered to fall within the scope of the invention so long as the polypeptide encodes a protein having p33^{QIK} activity. Such polypeptides may be deleted by one or more amino acid residues at either the amino terminus or the carboxy terminus and still contemplated to fall within the scope of the invention, so long as a measurable amount of this activity is retained by the substantially full length variant. (e.g., position 1 to position 321, position 1 to position 320, position 1 to position 319, position 1 to position 318, etc. all of SEQ ID NO:2).

The invention also encompasses polynucleotides that comprise at least a first sequence region that encodes one or more of the peptides or peptide variants as disclosed herein. Such polynucleotides may comprise a sequence region of 27 to about 5000 nucleotides in length, or a sequence region of 27 to about 2000 nucleotides in length, or a sequence region of 27 to about 1000 nucleotides in length, or a sequence region of 27 to about 900, or about 800, or about 700, or about 600, or about 500, or about 400, or about 300, or about 200, or even about 100 or so nucleotides in length.

As in the case of the peptides, the length of the sequence region that encodes the peptide may be of any intermediate length in these ranges, such as those polynucleotides that comprise at least a first sequence region of from about 30 to about 750 nucleotides in length, those that comprise at least a first sequence region of from about 35 to about 650 nucleotides in length, and those that comprise at least a first sequence region of from about 40 to about 550, about 450, about 350, about 250, about 150, or even about 50 or so nucleotides in length. Such sequence regions may be on the order of about 27, or about 28, or about 29, or about 30, or about 31, or about 32, or about 33, or about 34, or even about 35 or so nucleotides in length, so long as the sequence region encodes at least a first peptide that comprises at least a first contiguous amino acid sequence according to any one of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, as well as any one of SEQ ID NO:21 to SEQ ID NO:30, SEQ ID NO:31 to SEQ ID NO:40, SEQ ID NO:41 to SEQ ID NO:50, SEQ ID NO:51 to SEQ ID NO:60, SEQ ID NO:61 to SEQ ID NO:70, or SEQ ID NO:71 to SEQ ID NO:76, or from about amino acid 1 to amino acid 322 of SEQ ID NO:2. When intermediate-length antigenic peptides or antigen binding fragments are desired, the nucleic acids that encode them may be on the order of about 40, about 45, or about 50, or about 55, or about 60, or about 65, or about 70, or about 75, or about 80, or even about 85 or 90 or so nucleotides in length, so long as they each encode at least a first peptide that comprises at least a first contiguous amino acid sequence according to any one of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, as well as any one of SEQ ID NO:21 to SEQ ID NO:30, SEQ ID NO:31 to SEQ ID NO:40, SEQ ID NO:41 to SEQ ID NO:50, SEQ ID NO:51 to SEQ ID NO:60, SEQ ID NO:61 to SEQ ID NO:70, or SEQ ID NO:71 to SEQ ID NO:76, or from about amino acid 1 to amino acid 322 of SEQ ID NO:2. When polynucleotides are contemplated that comprise sequence regions encoding larger antigenic peptides or antigen-binding fragments, the nucleic acid sequence region encoding them will necessarily be longer in length. For example, a nucleic acid sequence region encoding a peptide or antigen binding fragment on the order of about 40 to 50 amino acids in length, will necessarily be at least from about 120 to about 150 or so nucleotides in length, given the fact that a triplet codon is required to encode a single amino acid.

Likewise, the polynucleotides comprising such sequence regions can be substantially larger than the coding region itself, particularly when the sequence region is operably linked to one or more promoters, or to one or more sequence regions that encode one or more signal sequences, and/or peptide fusion products. In those embodiments, the polynucleotide may be on the order of about 500, about 600, about 700, about 800, about 900, about 1000, about 1100, about 1200, about 1300, about 1400, or even about 1500, 1600, 1700, 1800, 1900, or even 2000 or so nucleotides in length, even up to and including those sequences that are on the order of about 10,000 or so nucleotides in length. Such polynucleotides are particularly useful in the preparation of expression vectors, delivery vehicles, viral vectors, and transformed host cells that express the particular encoded peptide(s) and/or antigen-binding fragment(s) encoded by the sequence region comprised within the polynucleotide and/or genetic construct or expression element.

In another exemplary embodiment, the peptide comprises at least a first isolated peptide of from 9 to about 11 amino acids in length, or at least a first nucleic acid segment that encodes the peptide; wherein the peptide consists essentially of the amino acid sequence of any one of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, as well as any one of SEQ ID NO:21 to SEQ ID NO:30, SEQ ID NO:31 to SEQ ID NO:40, SEQ ID NO:41 to SEQ ID NO:50, SEQ ID NO:51 to SEQ ID NO:60, SEQ ID NO:61 to SEQ ID NO:70, or SEQ ID NO:71 to SEQ ID NO:76, or from about amino acid 1 to amino acid 322 of SEQ ID NO:2.

Similarly, in another related embodiment, the peptide comprises at least a first isolated peptide of from 9 to about 10 or 11 or so amino acids in length, or at least a first nucleic acid segment that encodes the peptide; wherein the peptide consists of the amino acid sequence of any one of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, as well as any one of SEQ ID NO:21 to SEQ ID NO:30, SEQ ID NO:31 to SEQ ID NO:40, SEQ ID NO:41 to SEQ ID NO:50, SEQ ID NO:51 to SEQ ID NO:60, SEQ ID NO:61 to SEQ ID NO:70, or SEQ ID NO:71 to SEQ ID NO:76, or from about amino acid 1 to amino acid 322 of SEQ ID NO:2.

In addition to peptides and compositions that comprise a single peptide species, the invention also concerns compositions that comprise 2, 3, 4, or more peptide species and/or the polynucleotides that encode such peptides. Such pluralities of peptide and/or polynucleotide species are particularly desirable in the formulation of therapeutic agents that comprise pluralities of peptides having two or more different contiguous amino acid sequence as disclosed in the amino acid sequences of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20, as well as any one of SEQ ID NO:21 to SEQ ID NO:30, SEQ ID NO:31 to SEQ ID NO:40, SEQ ID NO:41 to SEQ ID NO:50, SEQ ID NO:51 to SEQ ID NO:60, SEQ ID NO:61 to SEQ ID NO:70, or SEQ ID NO:71 to SEQ ID NO:76, or from about amino acid 1 to amino acid 322 of SEQ ID NO:2, and/or a plurality of polynucleotides that encode such peptides. Irrespective of the source of the particular antigenic peptide and polynucleotide compounds, the invention particularly contemplates the use of one, two, three or four distinct peptides, polynucleotides or derivatives thereof, up to and including a plurality of such compounds. This exemplifies the use of singular terminology throughout the entire application,, wherein the terms “a” and “an” are used in the sense that they mean “at least one”, “at least a first”, “one or more” or “a plurality” of the referenced components or steps, except in instances wherein an upper limit is thereafter specifically stated or would be understood by one of ordinary skill in the art. The operable limits and parameters of combinations, as with the amounts of any single agent, will be known to those of ordinary skill in the art in light of the present disclosure.

The additional peptides in such compositions may all be of approximately the same size and/or approximately the same primary amino acid sequence, or alternatively, the peptides may differ considerably in length and/or primary amino acid sequence. Such compositions may further comprise one or more additional components, such as for example, a pharmaceutically acceptable excipient, buffer, or reagent as described in detail hereinbelow. Such compositions may also optionally further comprise at least a first immunostimulant or at least a first adjuvant as described herein. Such immunostimulants and adjuvants preferentially enhance a T-cell response in a human, and may preferably be selected from the group consisting of Montanide, a cytokine, a microsphere, Ribi Adjuvant, saponin, a microfluidized adjuvant, an immune stimulating complex and an inactivated toxin. As described in more detail hereinbelow, the compositions may be formulated for diagnostic or therapeutic uses, including their incorporation into one or more diagnostic or therapeutic kits for clinical packaging and/or commercial resale, with those formulations suitable for administration to a mammal, such as a human, with parenteral, intravenous, intraperitoneal, subcutaneous, intranasal, transdermal, and oral routes being particularly preferred.

The compositions may further optionally comprise one or more detection reagents, one or more additional diagnostic reagents, one or more control reagents, and/or one or more therapeutic reagents. In the case of diagnostic reagents, the compositions may further optionally comprise one or more detectable labels that may be used in both in vitro and/or in vivo diagnostic and therapeutic methodologies. In the case of therapeutic compositions and formulations, the compositions of the invention may also further optionally comprise one or more additional anti-cancer, or otherwise therapeutically-beneficial components as may be required in particular circumstances, and such like.

These and other aspects of the present invention will become apparent upon reference to the following detailed description and attached drawings. All references disclosed herein are hereby incorporated by reference in their entirety as if each was incorporated individually.

As noted above, the peptides of the present invention may comprise one or more variants of the amino acid sequences as disclosed herein. A peptide “variant,” as used herein, is a peptide that differs from a particular primary amino acid sequence in one or more substitutions, deletions, additions and/or insertions, such that the immunogenicity of the peptide is substantially retained (i.e., the ability of the variant to react with antigen-specific antisera and/or T-cell lines or clones is not substantially diminished relative to the native peptide). In other words, the ability of a variant to react with antigen-specific antisera and/or T-cell lines or clones may be enhanced or unchanged, relative to the peptide from which the variant was derived.

Preferably, the biological activity of a peptide variant will not be diminished by more than 1%, and preferably still will not be diminished by more than 2%, relative to the biological activity of the unmodified peptide. More preferably, the biological activity of a peptide variant will not be diminished by more than 3%, and more preferably still will not be diminished by more than 4%, 5%, 6%, 7%, 8%, or 9%, relative to the biological activity of the unmodified peptide. More preferably still, the biological activity of a peptide variant will not be diminished by more than 10%, and more preferably still, will not be diminished by more than 11%, 12%, 13%, 14%, 15%, 16%, 17%, 18%, 19%, or 20% relative to the biological activity of the corresponding unmodified peptide.

Based upon % sequence homology, preferred peptide variant of the present invention include those peptides that are from 9 to about 100 amino acids in length, and that comprise at least a first sequence region that is at least 75% identical to at least one of the amino acid sequences disclosed in any one of SEQ ID NO:3 through SEQ ID NO:79, and more preferably those that comprise at least a first sequence region that is at least 80% identical to at least one of the amino acid sequences disclosed in any one of SEQ ID NO:3 through SEQ ID NO:79. More preferably, based upon % sequence homology, preferred peptide variants of the present invention are those peptides that comprise at least a first sequence region that is at least 85% identical to at least one of the amino acid sequences disclosed in any one of SEQ ID NO:3 through SEQ ID NO:79, and more preferably those that comprise at least a first sequence region that is at least 90% identical to at least one of the amino acid sequences disclosed in any one of SEQ ID NO:3 through SEQ ID NO:79. Particularly preferred peptide variants of the present invention are those peptides that comprise at least a first sequence region that is at least 91%, 92%, 93%, 94%, or 95% identical to at least one of the amino acid sequences disclosed in any one of SEQ ID NO:3 through SEQ ID NO:79, with those peptides that comprise at least a first sequence region that is at least 96%, 97%, 98%, or 99% identical to at least one of the amino acid sequences disclosed in any one of SEQ ID NO:3 through SEQ ID NO:79.

Such peptide variants may typically be prepared by modifying one of the peptide sequences disclosed herein, and particularly by modifying the primary amino acid sequence of one or more of the peptide epitopes disclosed in any one of SEQ ID NO:3 through SEQ ID NO:79. These biological functional equivalent peptides may encompass primary amino acid sequences that differ from the original peptide sequences disclosed in any one of SEQ ID NO:3 through SEQ ID NO:79 by one or more conservative amino acid substitutions.

It has been found, within the context of the present invention, that a relatively small number of conservative or neutral substitutions (e.g., 1 or 2) may be made within the sequence of the peptide epitopes disclosed herein, without substantially altering the biological activity of the peptide. In some cases, the substitution of one or more amino acids in a particular peptide may in fact serve to enhance or otherwise improve the ability of the peptide to elicit an immune or T-cell response in an animal that has been provided with a composition that comprises the modified peptide, or a polynucleotide that encodes the peptide. Suitable substitutions may generally be identified by using computer programs, as described hereinbelow, and the effect of such substitutions may be confirmed based on the reactivity of the modified peptide with antisera and/or T-cells as described herein. Accordingly, within certain preferred embodiments, a peptide for use in the disclosed diagnostic and therapeutic methods may comprise a primary amino acid sequence in which one or more amino acid residues are substituted by one or more replacement amino acids, such that the ability of the modified peptide to react with antigen-specific antisera and/or T-cell lines or clones is not significantly less than that for the unmodified peptide. Exemplary such substitutions may preferably be located within one or more MHC binding sites on the peptide.

As described above, preferred peptide variants are those that contain one or more conservative substitutions. A “conservative substitution” is one in which an amino acid is substituted for another amino acid that has similar properties, such that one skilled in the art of peptide chemistry would expect the secondary structure and hydropathic nature of the peptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Examples of amino acid substitutions that represent a conservative change include: (1) replacement of one or more Ala, Pro, Gly, Glu, Asp, Gln, Asn, Ser, or Thr; residues with one or more residues from the same group; (2) replacement of one or more Cys, Ser, Tyr, or Thr residues with one or more residues from the same group; (3) replacement of one or more Val, Ile, Leu, Met, Ala, or Phe residues with one or more residues from the same group; (4) replacement of one or more Lys, Arg, or His residues with one or more residues from the same group; and (5) replacement of one or more Phe, Tyr, Trp, or His residues with one or more residues from the same group.

A variant may also, or alternatively, contain nonconservative changes, for example, by substituting one of the amino acid residues from group (1) with an amino acid residue from group (2), group (3), group (4), or group (5). Variants may also (or alternatively) be modified by, for example, the deletion or addition of amino acids that have minimal influence on the immunogenicity, secondary structure and hydropathic nature of the peptide.

2.3 Western Blots

The antibody compositions of the present invention will find great use in immunoblot or western blot analysis. The antibodies may be used as high-affinity primary reagents for the identification of proteins immobilized onto a solid support matrix, such as nitrocellulose, nylon or combinations thereof. In conjunction with immuno-precipitation, followed by gel electrophoresis, these may be used as a single step reagent for use in detecting antigens against which secondary reagents used in the detection of the antigen cause an adverse background. This is especially useful when the antigens studied are obtained from mammalian p33^{QIK} or p63^{Krsl} polypeptides.

Immunologically-based detection methods for use in conjunction with Western blotting include enzymatically-, radiolabel-, or fluorescently-tagged secondary antibodies against the p33^{QIK} or p63^{Krsl}-specific antigens are considered to be of particular use in this regard.

2.4 ELISAS and Immunoprecipitation

One important utility of the antibodies is in immunoassays for the detection and quantitation of p33^{QIK} or p63^{Krsl}-specific antibodies, as needed in diagnosis and prognostic monitoring.

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and Western blotting, dot blotting, FACS analyses, and the like may also be used.

In one exemplary ELISA, the antibodies of the invention are immobilized onto a selected surface exhibiting protein affinity, such as a well in a polystyrene microtiter plate. Then, a test composition suspected of containing p33^{QIK} or p63^{Krsl} polypeptides, such as a clinical sample, is added to the wells. After binding and washing to remove non-specifically bound immunecomplexes, the bound p33^{QIK} or p63^{Krsl} polypeptides may be detected. Detection is generally achieved by the addition of another anti-mouse antibody that is linked to a detectable label that has specificity for the primary anti- p33^{QIK} or p63^{Krsl} antibody. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing p33^{QIK} or p63^{Krsl} polypeptides are immobilized onto the well surface and then contacted with the antibodies of the invention. After binding and washing to remove non-specifically bound immunecomplexes, the bound antibodies are detected. Where the initial anti- p33^{QIK} or p63^{Krsl} antibodies are linked to a detectable label, the immunecomplexes may be detected directly. Again, the immunecomplexes may be detected using a second antibody that has binding affinity for the first antibody, with the second antibody being linked to a detectable label.

Another ELISA in which the polypeptides are immobilized, involves the use of antibody competition in the detection. In this ELISA, labeled antibodies are added to the wells, allowed to bind, and detected by means of their label. The amount of p33^{QIK} or p63^{Krsl} polypeptides in an unknown sample is then determined by mixing the sample with the labeled anti- p33^{QIK} or p63^{Krsl} antibodies before or during incubation with coated wells. The presence of p33^{QIK} or p63^{Krsl} polypeptides in the sample acts to reduce the amount of antibody available for binding to the well and thus reduces the ultimate signal. This is also appropriate for detecting similar antibodies in an unknown sample, where the unlabeled antibodies bind to the antigen-coated wells and also reduces the amount of antigen available to bind the labeled antibodies.

Competition ELISAs are also possible in which test samples compete for binding with known amounts of labeled p33^{QIK} or p63^{Krsl} polypeptides or p33^{QIK} or p63^{Krsl}-specific antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal.

Irrespective of the format employed, ELISAs have certain features in common, such as coating, incubating or binding, washing to remove non-specifically bound species, and detecting the bound immunecomplexes. These are described as follows:

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control p33^{QIK} or p63^{Krsl} polypeptide composition and/or clinical or biological sample to be tested under conditions effective to, or in a manner conducive to, allow immunecomplex (antigen/antibody) formation. Detection of the immunecomplex then requires a labeled secondary binding ligand or antibody, or a secondary binding ligand or antibody in conjunction with a labeled tertiary antibody or third binding ligand.

“Under conditions effective to” or “in a manner conducive to” allow immunecomplex (antigen/antibody) formation means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hr, at temperatures preferably on the order of about 25° to about 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immunecomplex with a urease, glucose oxidase, alkaline phosphatase or hydrogen peroxidase-conjugated antibody for a period of time and under conditions that favor the development of further immunecomplex formation (e.g., incubation for 2 hr at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azino-di-(3-ethyl-benzthiazoline-6-sulfonic acid [ABTS] and H₂O₂, in the case of peroxidase as the enzyme label. Quantification is then achieved by measuring the degree of color generation, e.g., using a visible spectra spectrophotometer.

2.5 Biological Functional Equivalents

Modification and changes may be made in the structure of the polynucleotides and peptides of the present invention and still obtain a functional molecule that encodes a peptide with desirable characteristics, or still obtain a genetic construct with the desirable expression specificity and/or properties. As it is often desirable to introduce one or more mutations into a specific polynucleotide sequence, various means of introducing mutations into a polynucleotide or peptide sequence known to those of skill in the art may be employed for the preparation of heterologous sequences that may be introduced into the selected cell or animal species. In certain circumstances, the resulting encoded peptide sequence is altered by this mutation, or in other cases, the sequence of the peptide is unchanged by one or more mutations in the encoding polynucleotide. In other circumstances, one or more changes are introduced into the promoter and/or enhancer regions of the polynucleotide constructs to alter the activity, or specificity of the expression elements and thus alter the expression of the heterologous therapeutic nucleic acid segment operably positioned under the control of the elements.

When it is desirable to alter the amino acid sequence of one or more of the heterologous peptides encoded by the expression construct to create an equivalent, or even an improved, second-generation molecules, the amino acid changes may be achieved by changing one or more of the codons of the encoding DNA sequence, according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions, or corresponding DNA sequences which encode said peptides without appreciable loss of their biological utility or activity.

TABLE 1

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporate herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those that are within ±1 are particularly preferred, and those within ±0.5 are even more particularly preferred. It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those that are within +1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take several of the foregoing characteristics into consideration are well known to those of skill in the art and include: arginine and lysine; glutamate and aspartate; serine and threonine; glutamine and asparagine; and valine, leucine and isoleucine.

The peptides and peptide variants of the present invention may be conjugated to a signal (or leader) sequence at the N-terminal end of the peptide, which co-translationally or post-translationally directs transfer of the peptide. The peptides may also, or alternatively, be conjugated to one or more linker sequences for ease of synthesis, purification or identification of the peptide (e.g., poly-His), or to enhance binding of the peptide to a solid support. For example, the peptides may be conjugated to an immunoglobulin Fc region.

The peptides and peptide variants of the present invention may be isolated and purified from native sources, such as for example, by isolating all or part of the primary amino acid sequence from a native peptide, or alternatively, may be chemically synthesized in whole or in part using any of a variety of well-known peptide synthesis techniques. For example, peptides having less than about 100 amino acids, preferably less than about 90 or 80 amino acids, and more preferably less than about 70, less than about 60, or less than about 50, about 40, about 30, or about 20 amino acids, may be synthesized using any of the commercially available solid-phase techniques, such as the Merrifield solid-phase synthesis method, where amino acids are sequentially added to a growing amino acid chain (Merrifield, 1963). Equipment for automated synthesis of peptides is commercially available from suppliers such as Applied BioSystems, Inc. (Foster City, Calif.), and may be operated according to the manufacturer's instructions.

The peptides and peptide variants as described herein may also be readily prepared from recombinant peptides, or may be prepared by translation of a polynucleotide sequence that encodes such a peptide. In general, any of a variety of expression vectors known to those of ordinary skill in the art may be employed to express recombinant peptides. Expression may be achieved in any appropriate host cell that has been transformed or transfected with an expression vector containing a nucleic acid molecule that encodes the peptide. Suitable host cells include prokaryotes, yeast and higher eukaryotic cells. Preferably, the host cells employed are E. coli, yeast or a mammalian cell line such as COS or CHO.

In general, peptides and polynucleotides as described herein are isolated. An “isolated” peptide or polynucleotide is one that is removed from its original environment. For example, a naturally occurring peptide or polypeptide is isolated if it is separated from some or all of the coexisting materials in the natural system. Preferably, such peptides are at least about 80% or 85% pure, more preferably at least about 90% or 95% pure and most preferably at least about 96%, 97%, 98%, or 99% pure. A polynucleotide is considered to be isolated if, for example, it is cloned into a vector that is not a part of the natural environment.

Within further aspects, the present invention provides mimetics of the peptides disclosed herein. Such mimetics may comprise amino acids linked to one or more amino acid mimetics (i.e., one or more amino acids within the p33^{QIK} protein may be replaced by an amino acid mimetic) or may be entirely mimetics. An amino acid mimetic is a compound that is conformationally similar to an amino acid such that it can be substituted for an amino acid within one or more of the disclosed peptides without substantially diminishing the ability to react with antigen-specific antisera and/or T cell lines or clones. A mimetic is a compound that does not contain amino acids, and that has an overall conformation that is similar to a p33^{QIK} peptide such that the ability of the mimetic to react with p33^{QIK} -specific antisera and/or T cell lines or clones is not substantially diminished relative to the ability of a p33^{QIK} peptide. Such mimetics may be designed based on standard techniques (e.g., nuclear magnetic resonance and computational techniques) that evaluate the three dimensional structure of a peptide sequence. Mimetics may be designed where one or more of the side chain functionalities of the p33^{QIK} peptide are replaced by groups that do not necessarily have the same size or volume, but have similar chemical and/or physical properties which produce similar biological responses. It should be understood that, within embodiments described herein, a mimetic may be substituted for a p33^{QIK} peptide.

Within other illustrative embodiments, a polypeptide may be a fusion polypeptide that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags, which facilitate purification of the polypeptide.

Fusion polypeptides may generally be prepared using standard techniques, including chemical conjugation. Preferably, a fusion polypeptide is expressed as a recombinant polypeptide, allowing the production of increased levels, relative to a non-fused polypeptide, in an expression system. Briefly, DNA sequences encoding the polypeptide components may be assembled separately, and ligated into an appropriate expression vector. The 3′-end of the DNA sequence encoding one polypeptide component is ligated, with or without a peptide linker, to the 5′-end of a DNA sequence encoding the second polypeptide component so that the reading frames of the sequences are in phase. This permits translation into a single fusion polypeptide that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., 1985; Murphy et al., 1986; U.S. Pat. No. 4,935,233 and U.S. Pat. No. 4,751,180. The linker sequence may generally be from 1 to about 10, about 20, about 30, about 40, or about 50 or so amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The fusion polypeptide can comprise a polypeptide as described herein together with an unrelated immunogenic protein, such as an immunogenic protein capable of eliciting a recall response. Examples of such proteins include tetanus, tuberculosis and hepatitis proteins (see, for example, Stoute et al., 1997).

### 3.0 BRIEF DESCRIPTION OF THE DRAWINGS

The drawings form part of the present specification and are included to further demonstrate certain aspects of the present invention. The invention may be better understood by reference to one or more of the following drawings in combination with the detailed description of specific embodiments presented herein:

FIG. 1A, FIG. 1B, FIG. 1C, FIG. 1D, FIG. 1E, FIG. 1F, and FIG. 1G show the regulation of p33^{QIK} activity in the cell cycle. NIH3T3 cells were arrested in quiescent state by serum starvation for 48 hr (FIG. 1A; FIG. 1G, lane 1), followed by serum stimulation to enter the cell cycle for 0.5 and 1 hr (FIG. 1G, lanes 2 and 3), 6 or 18 hr (FIG. 1B and FIG. 1C; FIG. 1G, lanes 4 and 5). After 18 hr of serum stimulation, cultures were serum-deprived and maintained in serum-free medium for 4 hr (FIG. 1G, lane 6), 6 hr (FIG. 1D), 8 hr (FIG. 1G, lane 7), 12 and 48 hr (FIG. 1E and FIG. 1F; FIG. 1G, lanes 8 and 9) for the second growth arrest, followed by serum stimulation to re-enter the cell cycle for 1 or 6 hr (FIG. 1G, lanes 10 and 11). FIG. 1A-FIG. 1F show flow cytometric analysis of DNA content to determine cell population in each phase of the cell cycle. FIG. 1G shows the kinase activities of p33^{QIK} and p44/42^{Erk} in 20 μg of cytosolic proteins (S20) were measured by the in-gel kinase assay using MBP as a substrate. Arrow indicates p33^{QIK}. Bars indicate p44/42^{Erk}.

FIG. 2A, FIG. 2B, and FIG. 2C show the activation of p33^{QIK} in cultures arrested in G0/G1 phase by staurosporine. NIH3T3 cultures were synchronized in quiescent state by serum starvation for 48 hr. Quiescent NIH3T3 cultures were treated with 5% serum to enter the cell cycle for 18 hr (FIG. 2B, lane 1). Cultures were then grown in medium in the absence (FIG. 2A; FIG. 2C, lane 2) or presence of 10 nM STSP (FIG. 2B; FIG. 2C, lane 3) for 24 hr. FIG. 2A-FIG. 2B show flow cytometric analysis of DNA content to determine cell population in each phase of the cell cycle. FIG. 2C shows the kinase activity of p33^{QIK} in 20 μg cytosolic proteins (S20) was analyzed by the in-gel kinase assay using MBP as a substrate. Arrow indicates p33^{QIK}

FIG. 3A, FIG. 3B, FIG. 3C, FIG. 3D, FIG. 3E, and FIG. 3F show the activation of p33^{QIK} in cells treated with FR. Proliferative NIH3T3 cultures (FIG. 3E; FIG. 3F, lane 5) were treated with 5 nM FR for 6, 12 or 24 hr (FIGS. 3A-FIG. 3C; FIG. 3F, lanes 1 to 3) or growth-arrested by serum starvation for 24 hr (FIG. 3D; FIG. 3F, lane 4). FIG. 3A-FIG. 3E show flow cytometric analysis of DNA content of cell population in each phase of the cell cycle. FIG. 3F shows the kinase activity of p33^{QIK} in 20 μg of cytosolic proteins (S20) was measured by the in-gel kinase assay using MBP as a substrate. Arrow indicates p33^{QIK}.

FIG. 4 shows the correlation between deactivation of p33^{QIK} and activation of p44/42^{Erk}. NIH3T3 cultures were arrested in quiescent state by serum starvation for 48 hr (lane 1), followed by stimulation with 10% serum (lanes 2 to 4) or 2 mM Na₃VO₄ (lanes 5 to 7) for 1 hr (lanes 2 and 5), 3 hr (lanes 3 and 6), and 6 hr (lanes 4 and 7). Protein kinases in 20 mg of cytosolic proteins (S20) were analyzed by the in-gel kinase assay using MBP as a substrate. Arrow indicates p33^{QIK} Bars indicate p44/42^{Erk}.

FIG. 5 shows the induction of p33^{QIK} activity by inhibition of macromolecular synthesis. NIH3T3 cultures were arrested in quiescent state by serum starvation for 48 hr (lane 1), followed by stimulation with 10% serum (lanes 2-7) or 100 ng/ml TPA (lanes 8-13) in the presence of 5 μg/ml ActD (lanes 4, 5, 10, 11, 14, and 15) or 10 μg/ml CHXM (lanes 6, 7, 12, 13, 16, and 17) for 1 hr (lanes 2, 4, 6, 8, 10, 12, 14, and 16) or 3 hr (lanes 3, 5, 7, 9, 11, 13, 15, and 17). Protein kinases in 20 μg cytosolic proteins (S20) were analyzed by the in-gel kinase assay using MBP as a substrate. Arrow indicates p33^{QIK}. Bars indicate p44/42^{Erk}.

FIG. 6A and FIG. 6B show the activation of p33^{QIK} in cell quiescence by stress shock. NIH3T3 cultures were synchronized in quiescent state by serum starvation for 48 hr (FIG. 6A, lanes 1 to 3; FIG. 6B, lanes 1 and 2). Quiescent cultures were stimulated with 10% serum for 1 or 20 hr for entry into G1 (FIG. 6A, lanes 4 and 5; FIG. 6B, lanes 3 and 4) or G2/M (FIG. 6A, lanes 6 and 7; FIG. 6B, lanes 5 and 6) phase of the cell cycle. FIG. 6A shows cultures were treated with 100 mJ/cm² of UV irradiation followed by incubation for 1 hr (lane 2) or 3 hr (lanes 3, 5, and 7). FIG. 6B for osmotic shock, cultures were treated with 300 mM NaCl for 2 hr (lanes 2, 4, and 6). The kinase activity of p33^{QIK} in 20 μg of cytosolic lysates (S20) was measured by the in-gel kinase assay using MBP as a substrate. Arrow indicates p33^{QIK}.

FIG. 7A, FIG. 7B, FIG. 7C, and FIG. 7D show Ab-KQ specific to p63^{Krsl} and p33^{QIK}. Growing NIH3T3 cultures (G) were synchronized in quiescent state by serum starvation for 48 hr (G0), followed by treatment with 300 mM NaCl (G0/NaCl) or 100 nM STSP (G0/STSP) for 2 hr (FIG. 7A). The kinase activities of p63^{Krsl} and p33^{QIK} in 20 μg of cytosolic lysates (S20) was measured by the in-gel kinase assay (FIG. 7B). Protein levels of p63^{Krsl} and p33^{QIK} in 40 μg of S20 were determined by western immunoblotting with Ab-KQ (lanes 1 to 4) or Ab-KQ preincubated with antigen peptides (Ab-KQ+Ag)(lanes 5 to 8) (FIG. 7C). The kinase activities of p63^{Krsl} and p33^{QIK} that were prepared from 20 μg of S20 immuno-precipitated (IP) with 1 μl (odd lanes) or 5 μl (even lanes) of Ab-KQ were determined by the in-gel kinase assay (FIG. 7D). The kinase activities of p63^{Krsl} and p33^{QIK} in 10 μg of S20 (lanes 1 and 4) and in the immune complexes prepared from 10 μg of S20 incubated with 1 μl of Ab-KQ (IP)(lanes 2 and 5) in the presence of 1 μg of antigen peptides (IP+Ag)(lanes 3 and 6) were determined by the in-gel kinase assay. MBP was used as a substrate in the in-gel kinase assay. Bars indicate p63^{Krsl} and p33^{QIK}.

FIG. 8 shows Caspase-3 in modification of p63^{Krsl} and p33^{QIK} Growing NIH3T3 cultures were treated with 20 nM STSP for 24 hr (G/STSP) or synchronized in quiescent state by serum starvation for 48 hr followed by treatment with 300 mM NaCl (G0/NaCl) for 2 hr. Both p63^{Krsl} and p33^{QIK} in 10 μg of cytosolic lysates (S20) were immunoprecipitated by Ab-KQ (lanes 2 to 4 and 6 to 8), followed by reatment with 0.2 μg Caspase-3 (C)(lanes 3, 4, 7 and 8) in the presence of 5 μM caspase inhibitor Z-D-CH₂-DCB (C+I)(lanes 4 and 8) at 37° C. for 30 min. The kinase activities of p63^{Krsl}, p35, and p33^{QIK} in 5 μg of S20 (lanes 1 and 5) and in the immune complexes were determined by the in-gel kinase assay using MBP as a substrate. Bars indicate p63^{Krsl} p35, and p33^{QIK}.

FIG. 9 shows the deactivation of p33^{QIK} by phosphatase. NIH3T3 cultures were synchronized in quiescent state by serum starvation for 48 hr (G0), followed by treatment with 300 mM NaCl (G0/NaCl) for 2 hr. p33^{QIK} in 10 μg of cytosolic lysates (S20) were immunoprecipitated by Ab-KQ (IP), followed by incubation of the immune complexes with 0.1 unit of PP1 (Ps) (lanes 2, 3, 5 and 6) in phosphatase buffer (50 mM Tris-HCl, pH 7.0, 0.1 mM EDTA, 5 mM dithiothreitol, 0.01% Brij 35, 2 mM MnCl₂) in the presence of 10 mM Na₃VO₄ and 50 mM NaF (Ps+I)(lanes 3 and 6) at 30° C. for 30 min. The kinase activity of p33^{QIK} in the immune complexes was determined by the in-gel kinase assay using MBP as a substrate. Bar indicates p33^{QIK}.

FIG. 10 shows the activation of p33^{QIK}. Both p63^{Krsl} and p33^{QIK} in 50 mg of cytosolic proteins (S20) obtained from proliferative NIH3T3 cells (G)(lanes 1 to 4), quiescent (G0)(lanes 5 to 8), and quiescent cultures treated with 300 mM NaCl (G0/NaCl)(lanes 9 to 12) were immunoprecipitated by 1 μl of Ab-KQ (IP). The immune complexes were incubated with lysis buffer (−)(lanes 1, 5, and 9) or 10 μg of S20 prepared from cells of G (lanes 2, 6, and 10), G0(lanes 3, 7, and 11), or G0/NaCl (lanes 4, 8, and 12) in the presence of 50 μM γATP at 30° C. for 20 min, followed by washes with lysis buffer. The kinase activities of p63^{Krsl} and p33^{QIK} in the immune complexes were determined by the in-gel kinase assay using MBP as a substrate. Bars indicate p63^{Krsl} and p33^{QIK}.

FIG. 11A and FIG. 11B show Human p63^{Krsl} /SAMK gene (SEQ ID NO:1) and encoded protein sequence (SEQ ID NO:2).

### 4.0 DESCRIPTION OF ILLUSTRATIVE EMBODIMENTS

The invention lends itself to a multitude of practical applications, including the following:

Basic studies such as in the area of anticancer therapeutics, stress, and aging related to cell death Activation and protein formation of p33/36 have been ubiquitously detected in different types of cells undergoing apoptosis induced either by stress or an apoptosis inducer. Availability of an optimal specific antibody to Krsl and p33/36 will assist in identifying molecular mechanisms for apoptosis-related events. Anticancer agent-induced cell death in therapeutics or aging-related issues such as Alzheimer disease and neurodegeneration can be studied by taking advantage of availability of this high quality of antibody in the studies.

Preclinical studies relating to sensitivity of different organs and tissues to anticancer agents can be studied to determine toxicity of the agent to different organs by detecting p33/36 activity and protein formation in combination with histopathologic results.

Clinical studies for determining activation of p33/36 as an index for efficacy of anticancer agents may be conducted and the development of therapeutic testing kits. Since p33/36 can be detected in lymphoid cells undergoing apoptosis induced by anticancer agents, activation of p33/36 can be recognized as an index for effectivity of anticancer agent in clinical studies. White blood cells isolated from patients prior to and following chemotherapy will be lysed and analyzed by kinase assay and western immunoblotting to determine a) formation of p33/36 and b) activation of specific activity of p33/36. By comparing samples from patients pre- and post-chemotherapy, the molecular effect of an anticancer drug on induction of p33/36 during apoptosis can be determined. Induction of p33/36 may act as a molecular index of drug effectiveness in different phases of clinical trials.

Testing kits to determine both kinase activity and protein level in tissues, including isolation of cell lysates, activity assay, and protein assay using the specific antibody to Krsl and p33/36 are also contemplated to be within the scope of the invention.

4.1 Significance Of Krsl and its Derivatives p33/36

The Krsl gene encodes a protein kinase (Wang and Erikson, 1992; Taylor et al., 1996). Although the gene product consists of 491 amino acid residues, the denatured polypeptide behaves as a protein of approximately 63 kDa when detected by SDS-polyacrylamide gel electrophoresis.

p63^{Krsl} kinase activity can be detected in neuron, liver, lung, spleen, thymus, fibroblasts, and epithelial cells. It can also be detected in different species of animal including human, monkey, mouse, chicken, and xenopus. p63^{Krsl} shares 30% homology with Ste20p family which is involved in signaling control related to cell morphological changes.

Several groups have also reported that a protein kinase of 33 or 36 kDa is activated in cancer cells following treatment with anticancer agents and the p33/36 MBPK is a proteolytic product of p63^{Krsl} . Anticancer agents reported to induce apoptosis of cancerous cells and activation of p33/36 include FR901228 (Rajgolikar et al., 1998) and cytorienin A (Kakeya et al., 1998).

Activation of p33/36 is detected in cells undergoing apoptosis induced by UV-irradiation (Lu et al., 1996) and apoptosis inducers such as MT-21 (Watabe et al., 1999) and staurosporine (Graves et al., 1998). The inventor has demonstrated that detection of p33/36 activity can be utilized as a therapeutic efficacy marker in anticancer clinical studies using different anticancer drugs. In addition, the disclosed work indicates that activation of p33/36 is likely involved in signaling control for growth-arrest of normal cells in quiescent phase of the cell cycle and apoptotic process for malignantly transformed cells.

While investigating the signaling control regulated by protein kinases during the transition between G0 and G1 phases, a novel cytoplasmic protein kinase of 33 kDa (p33^{QIK}) was detected using the in-gel kinase assay with myelin basic protein (MBP) as a substrate. On the basis of p33^{QIK} activation in cultures arrested in quiescent state and its deactivation in cultures entering into G1 phase of the cell cycle, it appears that p33^{QIK} may be involved in signaling control for cell quiescence. Deactivation of p33 ^{QIK} appears to require expression of the immediate early G1 gene products, possibly protein phosphatases. Stress shock may additively increase p33^{QIK} activity correlated with induction of apoptosis in cultures arrested in the quiescent state but not in the other phases of the cell cycle.

Krsl-NC (lots #3105 and 3106) antiserum was obtained from rabbits immunized with a synthetic peptide with sequence of residues 296 to 311 of p63^{Krsl} , which is encoded by the Krsl gene (kinases responsive to stress). Because of the findings by several groups that a protein kinase of 33 or 36 kDa is activated in cancer cells following treatment with anticancer agents and the p33/36 MBPK is a proteolytic product of p63^{Krsl} , detection of p33/36 activity is highly potential to be utilized as a therapeutic efficacy market in anticancer clinical studies using different anticancer drugs. In addition, results of these studies indicate that activation of p33/36 may be involved in signaling control for growth-arrest of normal cells in quiescent phase of the cell cycle and apoptotic process for malignantly transformed cells. Since the Krsl-NC antibody is able to immunoprecipitate both p63^{Krsl} and p33/36 and none of the commercially available antibodies is able to immunoprecipitate p63^{Krsl} or p33/36, the Krsl-NC antibody should have a great practical value in basic and clinical studies of p63^{Krsl} and p33/36.

Normal and transformed cells that were treated with a natural anticancer agent FR901228 resulted in induction of p33 kinase activity. FR901228 has been evaluated as a single therapy or in combination with other agents in two-phase 1 clinical trials. FR901228 was isolated from the fermentation broth of Chromobacterium violaceum No. 968 and identified as an antitumor agent through an effort in the search for novel agents that selectively reversed the morphological phenotype of Ras oncogene-transformed cells. Induction of Ras activity has been detected in association with many human tumors such as breast tumors and expression of activated Ras results in induction of cancer-related features of transformed cells as seen in tumor progression. Treatment of Ras-transformed cells with FR901228 reversed morphologic transformation and inhibited RAS-induced myc expression, indicating that FR901228 blocked the Ras-induced mitogen-activated signaling pathway. In addition, FR901228 was found to act as an inhibitor to histone deacctylasc. FR901228 appears to have multiple molecular targets for its anticancer activity. Since the kinase activity of p33 is induced in cells following the treatment with FR901228, detection of p33 activity can be used as an index for the efficacy of FR901228 treatment in clinical trials and future chemotherapy. In addition, several groups also observed activation of p33/36 in their anticancer studies using different anticancer agents. Investigators may use general and conventional methods to determine the protein level and kinase activity of p33/36 by this specific antibody in assays, such as western immunoblotting, immunoprecipitation followed by kinase assay, and immunohistochemistry.

4.2 Antibody Compositions and Methods for their Production

In particular embodiments, the inventor contemplates the use of antibodies, either monoclonal or polyclonal that specifically bind to a mammalian p33^{QIK} or p63^{Krsl} polypeptide, and particularly those that specifically bind to one or more of the peptide sequences disclosed in any one of SEQ ID NO:3 through SEQ ID NO:76. Means for preparing and characterizing antibodies are well known in the art (See, e.g, Harlow and Lane, 1988; incorporated herein by reference). The methods for generating monoclonal antibodies (mAbs) generally begin along the same lines as those for preparing polyclonal antibodies. Briefly, a polyclonal antibody is prepared by immunizing an animal with an immunogenic composition in accordance with the present invention and collecting antisera from that immunized animal. A wide range of animal species can be used for the production of antisera. Typically the animal used for production of anti-antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As is also well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified tumor suppressor protein, polypeptide or peptide. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986, pp. 60-61), but mice are preferred, with the BALB/c mouse being most preferred as this is most routinely used and generally gives a higher percentage of stable fusions.

Following immunization, somatic cells with the potential for producing antibodies, specifically B lymphocytes (B cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (vol./vol.) PEG, (Gefter et al., 1977). The use of electrically induced fusion methods is also appropriate (Goding, 1986, pp. 71-74).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three wk) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

4.3 Polynucleotide Compositions

Any polynucleotide that encodes a p63^{Krsl}-derived peptide as described herein, or that is complementary to such a polynucleotide, is encompassed by the present invention. Such polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials. p63^{Krsl}-derived polynucleotides may encode a native p63^{Krsl}-derived epitopic protein, or may encode a variant of p63^{Krsl}-derived peptides as described herein. Polynucleotide variants may contain one or more substitutions, additions, deletions and/or insertions such that the immunogenicity of the encoded peptide is not diminished, relative to a native p63^{Krsl} protein. The effect on the immunogenicity of the encoded peptide may generally be assessed as described herein. Preferred peptide variants contain amino acid substitutions, deletions, insertions and/or additions at no more than about 20%, more preferably at no more than about 15%, and more preferably still, at no more than about 10% or 5% or less of the amino acid positions relative to the corresponding native unmodified p63^{Krsl}-derived amino acid sequence.

Likewise, polynucleotides encoding such peptide variants should preferably contain nucleotide substitutions, deletions, insertions and/or additions at no more than about 20%, more preferably at no more than about 15%, and more preferably still, at no more than about 10% or 5% or less of the nucleotide positions relative to the corresponding polynucleotide sequence that encodes the native unmodified p63^{Krsl}-derived peptide sequence. Certain polynucleotide variants, of course, may be substantially homologous to, or substantially identical to the corresponding region of the nucleotide sequence encoding an unmodified peptide. Such polynucleotide variants are capable of hybridizing to a naturally occurring DNA sequence encoding a p63^{Krsl}-derived peptide (or a complementary sequence) under moderately stringent, to highly stringent, to very highly stringent conditions.

Suitable moderately stringent conditions include prewashing in a solution containing about 5×SSC, 0.5% SDS, 1.0 mM EDTA (pH 8.0); hybridizing at a temperature of from about 50° C. to about 60° C. in 5×SSC overnight; followed by washing twice at about 60 to 65° C. for for 20 min. with each of 2×, 0.5× and 0.2×SSC containing 0.1% SDS). Suitable highly stringent conditions include prewashing in a solution containing about 5×SSC, 0.5% SDS, 1.0 mM EDTA (pH 8.0); hybridizing at a temperature of from about 60° C. to about 70° C. in 5×SSC overnight; followed by washing twice at about 65 to 70° C. for 20 min. with each of 2×, 0.5× and 0.2×SSC containing 0.1% SDS). Representative examples of very highly stringent hybridization conditions may include, for example, prewashing in a solution containing about 5×SSC, 0.5% SDS, 1.0 mM EDTA (pH 8.0); hybridizing at a temperature of from about 70° C. to about 75° C. in 5×SSC overnight; followed by washing twice at about 70° C. to about 75° C. for 20 min. with each of 2×, 0.5× and 0.2×SSC containing 0.1% SDS). Such hybridizing DNA sequences are also within the scope of this invention.

It will be appreciated by those of ordinary skill in the art that, as a result of the degeneracy of the genetic code, there are many nucleotide sequences that encode a p63^{Krsl}-derived peptide. Some of these polynucleotides bear minimal homology to the nucleotide sequence of any native gene. Nonetheless, polynucleotides that vary due to differences in codon usage are specifically contemplated by the present invention. p63^{Krsl}-derived peptide-encoding polynucleotides may also be synthesized by any method known in the art, including chemical synthesis (e.g., solid phase phosphoramidite chemical synthesis). Modifications in a polynucleotide sequence may also be introduced using standard mutagenesis techniques, such as oligonucleotide-directed site-specific mutagenesis (Adelman et al., 1983). Alternatively, RNA molecules may be generated by in vitro or in vivo transcription of DNA sequences encoding a p63^{Krsl}-derived peptide, provided that the DNA is incorporated into a vector with a suitable RNA polymerase promoter (such as T7 or SP6). Certain portions may be used to prepare an encoded peptide, as described herein. In addition, or alternatively, a portion may be administered to a patient such that the encoded peptide is generated in vivo (e.g., by transfecting antigen-presenting cells such as dendritic cells with a cDNA construct encoding a p63^{Krsl}-derived peptide, and administering the transfected cells to the patient).

Polynucleotides that encode a p63^{Krsl}-derived peptide may generally be used for production of the peptide, in vitro or in vivo. p63^{Krsl}-encoding polynucleotides that are complementary to a coding sequence (ie., antisense polynucleotides) may also be used as a probe or to inhibit p63^{Krsl} expression. cDNA constructs that can be transcribed into antisense RNA may also be introduced into cells of tissues to facilitate the production of antisense RNA.

Any polynucleotide may be further modified to increase stability in vivo. Possible modifications include, but are not limited to, the addition of flanking sequences at the 5′ and/or 3′-ends; the use of phosphorothioate or 2′-o-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Nucleotide sequences as described herein may be joined to a variety of other nucleotide sequences using established recombinant DNA techniques. For example, a polynucleotide may be cloned into any of a variety of cloning vectors, including plasmids, phagemids, lambda phage derivatives and cosmids. Vectors of particular interest include expression vectors, replication vectors, probe generation vectors, and sequencing vectors. In general, a vector will contain an origin of replication functional in at least one organism, convenient restriction endonuclease sites and one or more selectable markers. Other elements will depend upon the desired use, and will be apparent to those of ordinary skill in the art.

Within certain embodiments, polynucleotides may be formulated so as to permit entry into a cell of a mammal, and expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, or vaccinia or other poxvirus (e.g., avian poxvirus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art.

4.4 Methods of Nucleic Acid Delivery and DNA Transfection

In certain embodiments, it is contemplated that one or more RNA or DNA and/or substituted polynucleotide compositions disclosed herein will be used to transfect an appropriate host cell. Technology for introduction of RNAs and DNAs, and vectors comprising them into suitable host cells is well known to those of skill in the art. In particular, such polynucleotides may be used to genetically transform one or more host cells, when therapeutic administration of one or more active peptides, compounds or vaccines is achieved through the expression of one or more polynucleotide constructs that encode one or more therapeutic compounds of interest.

A variety of means for introducing polynucleotides and/or polypeptides into suitable target cells is known to those of skill in the art. For example, when polynucleotides are contemplated for delivery to cells, several non-viral methods for the transfer of expression constructs into cultured mammalian cells are available to the skilled artisan for his use. These include, for example, calcium phosphate precipitation (Graham and Van Der Eb, 1973; Chen and Okayama, 1987; Rippe et al., 1990); DEAE-dextran precipitation (Gopal, 1985); electroporation (Wong and Neumann, 1982; Fromm et al., 1985; Tur-Kaspa et al., 1986; Potter et al., 1984; Suzuki et al., 1998; Vanbever et al., 1998), direct microinjection (Capecchi, 1980; Harland and Weintraub, 1985), DNA-loaded liposomes (Nicolau and Sene, 1982; Fraley et al., 1979; Takakura, 1998) and lipofectamine-DNA complexes, cell sonication (Fechheimer et al., 1987), gene bombardment using high velocity microprojectiles (Yang et al., 1990; Klein et al., 1992), and receptor-mediated transfection (Curiel et al., 1991; Wagner et al., 1992; Wu and Wu, 1987; Wu and Wu, 1988). Some of these techniques may be successfully adapted for in vivo or ex vivo use.

A bacterial cell, a yeast cell, or an animal cell transformed with one or more of the disclosed expression vectors represent an important aspect of the present invention. Such transformed host cells are often desirable for use in the expression of the various DNA gene constructs disclosed herein. In some aspects of the invention, it is often desirable to modulate, regulate, or otherwise control the expression of the gene segments disclosed herein. Such methods are routine to those of skill in the molecular genetic arts. Typically, when increased or over-expression of a particular gene is desired, various manipulations may be employed for enhancing the expression of the messenger RNA, particularly by using an active promoter, and in particular, a tissue-specific promoter such as those disclosed herein, as well as by employing sequences, which enhance the stability of the messenger RNA in the particular transformed host cell.

Typically, the initiation and translational termination region will involve stop codon(s), a terminator region, and optionally, a polyadenylation signal. In the direction of transcription, namely in the 5′ to 3′ direction of the coding or sense sequence, the construct will involve the transcriptional regulatory region, if any, and the promoter, where the regulatory region may be either 5′ or 3′ of the promoter, the ribosomal binding site, the initiation codon, the structural gene having an open reading frame in phase with the initiation codon, the stop codon(s), the polyadenylation signal sequence, if any, and the terminator region. This sequence as a double strand may be used by itself for transformation of a microorganism or eukaryotic host, but will usually be included with a DNA sequence involving a marker, where the second DNA sequence may be joined to the expression construct during introduction of the DNA into the host.

Where no functional replication system is present, the construct will also preferably include a sequence of at least about 30 or about 40 or about 50 basepairs (bp) or so, preferably at least about 60, about 70, about 80, or about 90 to about 100 or so bp, and usually not more than about 500 to about 1000 or so bp of a sequence homologous with a sequence in the host. In this way, the probability of legitimate recombination is enhanced, so that the gene will be integrated into the host and stably maintained by the host. Desirably, the regulatory regions of the expression construct will be in close proximity to (and also operably positioned relative to) the selected therapeutic gene providing for complementation as well as the gene providing for the competitive advantage. Therefore, in the event that the therapeutic gene is lost, the resulting organism will be likely to also lose the gene providing for the competitive advantage, so that it will be unable to compete in the environment with the gene retaining the intact construct.

The selected therapeutic gene can be introduced between the transcriptional and translational initiation region and the transcriptional and translational termination region, so as to be under the regulatory control of the initiation region. This construct may be included in a plasmid, which will include at least one replication system, but may include more than one, where one replication system is employed for cloning during the development of the plasmid and the second replication system is necessary for functioning in the ultimate host, in this case, a mammalian host cell. In addition, one or more markers may be present, which have been described previously. Where integration is desired, the plasmid will desirably include a sequence homologous with the host genome.

Genes or other nucleic acid segments, as disclosed herein, can be inserted into host cells using a variety of techniques that are well known in the art. Five general methods for delivering a nucleic segment into cells have been described: (1) chemical methods (Graham and VanDerEb, 1973); (2) physical methods such as microinjection (Capecchi, 1980), electroporation (U.S. Pat. No. 5,472,869; Wong and Neumann, 1982; Fromm et al, 1985), microprojectile bombardment (U.S. Pat. No. 5,874,265, specifically incorporated herein by reference in its entirety), “gene gun” (Yang et al, 1990); (3) viral vectors (Eglitis and Anderson, 1988); (4) receptor-mediated mechanisms (Curiel et al., 1991; Wagner et al., 1992); and (5) bacterial-mediated transformation.

4.5 Administration of Pharmaceutical Compositions and Formulations

In certain embodiments, the present invention concerns formulation of one or more of the polynucleotide compositions disclosed herein in pharmaceutically acceptable solutions for administration to a cell or an animal, either alone, or in combination with one or more other modalities of anti-cancer therapy.

It will also be understood that, if desired, the nucleic acid segment, RNA, or DNA compositions disclosed herein may be administered in combination with other agents as well, such as, e.g., proteins or peptides or various pharmaceutically-active agents. As long as the composition comprises at least one of the genetic expression constructs disclosed herein, there is virtually no limit to other components that may also be included, given that the additional agents do not cause a significant adverse effect upon contact with the target cells or host tissues. The RNA- or DNA-derived compositions may thus be delivered along with various other agents as required in the particular instance. Such RNA or DNA compositions may be purified from host cells or other biological sources, or alternatively may be chemically synthesized as described herein. Likewise, such compositions may comprise substituted or derivatized RNA or DNA compositions. Such compositions may include one or more therapeutic gene constructs, either alone, or in combination with one or more modified peptide or nucleic acid substituent derivatives, and/or other anticancer therapeutics.

The formulation of pharmaceutically-acceptable excipients and carrier solutions are well-known to those of skill in the art, as is the development of suitable dosing and treatment regimens for using the particular compositions described herein in a variety of treatment regimens, including e.g., oral, intravenous, intranasal, transdermal, intraprostatic, intratumoral, and/or intramuscular administration and formulation.

4.5.1 Injectable Delivery

For example, the pharmaceutical compositions disclosed herein may be administered parenterally, intravenously, intramuscularly, or even intraperitoneally as described in U.S. Pat. No. 5,543,158, U.S. Pat. No. 5,641,515 and U.S. Pat. No. 5,399,363 (each specifically incorporated herein by reference in its entirety). Solutions of the active compounds as free-base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, Hoover, 1975). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

Sterile injectable solutions may be prepared by incorporating the gene therapy constructs in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

4.5.2 Intranasal Delivery

One may use nasal solutions or sprays, aerosols or even inhalants for therapy with one of more of the disclosed antibodies, peptides polypeptides and/or polynucleotides. Nasal solutions are usually aqueous solutions designed to be administered to the nasal passages in drops or sprays. Nasal solutions are prepared so that they are similar in many respects to nasal secretions, so that normal ciliary action is maintained. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known.

Inhalations and inhalants are pharmaceutical preparations designed for delivering a drug or compound into the respiratory tree of a patient. A vapor or mist is administered and reaches the affected area, often to give relief from symptoms of bronchial and nasal congestion. However, this route can also be employed to deliver agents into the systemic circulation. Inhalations may be administered by the nasal or oral respiratory routes. The administration of inhalation solutions is only effective if the droplets are sufficiently fine and uniform in size so that the mist reaches the bronchioles.

Another group of products, also known as inhalations, and sometimes called insufflations, consists of finely powdered or liquid drugs that are carried into the respiratory passages by the use of special delivery systems, such as pharmaceutical aerosols, that hold a solution or suspension of the drug in a liquefied gas propellant. When released through a suitable valve and oral adapter, a metered does of the inhalation is propelled into the respiratory tract of the patient.

Particle size is of importance in the administration of this type of preparation. It has been reported that the optimum particle size for penetration into the pulmonary cavity is of the order of 0.5 to 7 μm. Fine mists are produced by pressurized aerosols and hence their use in considered advantageous.

4.5.3 Liposome-, Nanocapsule-, and Microparticle-Mediated Delivery

In certain embodiments, the inventors contemplate the use of liposomes, nanocapsules, microparticles, microspheres, lipid particles, vesicles, and the like, for the introduction of the polynucleotide compositions of the present invention into suitable host cells. In particular, the polynucleotide compositions of the present invention may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids disclosed herein. The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-lives (Gabizon and Papahadjopoulos, 1988; Allen and Choun, 1987; U.S. Pat. No. 5,741,516, specifically incorporated herein by reference in its entirety). Further, various methods of liposome and liposome like preparations as potential drug carriers have been reviewed (Takakura, 1998; Chandran et al., 1997; Margalit, 1995; U.S. Pat. Nos. 5,567,434; 5,552,157; 5,565,213; 5,738,868 and U.S. Pat. No. 5,795,587, each specifically incorporated herein by reference in its entirety).

Liposomes have been used successfully with a number of cell types that are normally resistant to transfection by other procedures including T cell suspensions, primary hepatocyte cultures and PC12 cells (Renneisen et al., 1990; Muller et al., 1990). In addition, liposomes are free of the DNA length constraints that are typical of viral-based delivery systems. Liposomes have been used effectively to introduce genes, drugs (Heath and Martin, 1986; Heath et al., 1986; Balazsovits et al., 1989; Fresta and Puglisi, 1996), radiotherapeutic agents (Pikul et al., 1987), enzymes (Imaizumi et al., 1990a; Imaizumi et al., 1990b), viruses (Faller and Baltimore, 1984), transcription factors and allosteric effectors (Nicolau and Gersonde, 1979) into a variety of cultured cell lines and animals. In addition, several successful clinical trails examining the effectiveness of liposome-mediated drug delivery have been completed (Lopez-Berestein et al., 1985a; 1985b; Coune, 1988; Sculier et al., 1988). Furthermore, several studies suggest that the use of liposomes is not associated with autoimmune responses, toxicity or gonadal localization after systemic delivery (Mori and Fukatsu, 1992).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation.

In addition to the teachings of Couvreur et al. (1977; 1988), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars, and drugs.

Alternatively, the invention provides for pharmaceutically acceptable nanocapsule formulations of the polynucleotide compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made, as described (Couvreur et al., 1980; 1988; zur Muhlen et al., 1998; Zambaux et al. 1998; Pinto-Alphandry et al., 1995 and U.S. Pat. No. 5,145,684, specifically incorporated herein by reference in its entirety). In particular, methods of polynucleotide polynucleotide delivery to a target cell using either nanoparticles or nanospheres (Schwab et al., 1994; Truong-Le et al., 1998) are also particularly contemplated to be useful in formulating the disclosed compositions for administration to an animal, and to a human in particular.

4.6 Therapeutic Agents and Kits

The invention also provides one or more of the antibodies or antigen binding fragments, or peptides or peptide of the present invention variants formulated with one or more pharmaceutically acceptable excipients, carriers, diluents, adjuvants, and/or other components for administration to an animal in need thereof. In addition to the disclosed epitopes, antibodies and antigen binding fragments, antibody- or antigen binding fragment-encoding polynucleotides or additional therapeutic agents, polynucleotides, peptides, antigens, or other compounds as may be employed in the formulation of particular compositions and formulations disclosed herein, and particularly in the preparation of anticancer agents or therapeutics for administration to the affected mammal.

As such, preferred animals for administration of the pharmaceutical compositions disclosed herein include mammals, and particularly humans. Other preferred animals include primates, sheep, goats, bovines, equines, porcines, lupines, canines, and felines, as well as any other mammalian species commonly considered pets, livestock, or commercially relevant animal species. The compositions and formulations may include partially or significantly purified polypeptide, polynucleotide, or antibody or antigen binding fragment compositions, either alone, or in combination with one or more additional active ingredients, anticancer agents, vaccines, adjuvants, or other therapeutics which may be obtained from natural or recombinant sources, or which may be obtainable naturally or either chemically synthesized, or alternatively produced in vitro from recombinant host cells expressing one or more nucleic acid segments that encode one or more such additional active ingredients, carriers, adjuvants, cofactors, or other therapeutic compound.

4.7 Diagnostic Reagents and Kits

The invention further provides diagnostic reagents and kits comprising one or more such reagents for use in a variety of diagnostic assays, including for example, immunoassays such as ELISA and “sandwich”-type immunoassays. Such kits may preferably include at least a first peptide, or a first antibody or antigen binding fragment of the invention, a functional fragment thereof, or a cocktail thereof, and means for signal generation. The kit's components may be pre-attached to a solid support, or may be applied to the surface of a solid support when the kit is used. The signal generating means may come pre-associated with an antibody of the invention or may require combination with one or more components, e.g., buffers, antibody-enzyme conjugates, enzyme substrates, or the like, prior to use. Kits may also include additional reagents, e.g., blocking reagents for reducing nonspecific binding to the solid phase surface, washing reagents, enzyme substrates, and the like. The solid phase surface may be in the form of microtiter plates, microspheres, or other materials suitable for immobilizing proteins, peptides, or polypeptides. Preferably, an enzyme that catalyzes the formation of a chemiluminescent or chromogenic product or the reduction of a chemiluminescent or chromogenic substrate is a component of the signal generating means. Such enzymes are well known in the art.

Such kits are useful in the detection, monitoring and diagnosis of conditions characterized by over-expression or inappropriate expression of one or more of the disclosed peptides and/or polypeptides.

The therapeutic and diagnostic kits of the present invention may also be prepared that comprise at least one of the antibody, peptide, antigen binding fragment, hybridoma, vector, vaccine, polynucleotide, or cellular compositions disclosed herein and instructions for using the composition as a diagnostic reagent or therapeutic agent. Containers for use in such kits may typically comprise at least one vial, test tube, flask, bottle, syringe or other suitable container, into which one or more of the diagnostic and/or therapeutic composition(s) may be placed, and preferably suitably aliquoted. Where a second therapeutic agent is also provided, the kit may also contain a second distinct container into which this second diagnostic and/or therapeutic composition may be placed. Alternatively, a plurality of compounds may be prepared in a single pharmaceutical composition, and may be packaged in a single container means, such as a vial, flask, syringe, bottle, or other suitable single container. The kits of the present invention will also typically include a means for containing the vial(s) in close confinement for commercial sale, such as, e.g., injection or blow-molded plastic containers into which the desired vial(s) are retained. Where a radiolabel, chromogenic, fluorogenic, or other type of detectable label or detecting means is included within the kit, the labeling agent may be provided either in the same container as the diagnostic or therapeutic composition itself, or may alternatively be placed in a second distinct container means into which this second composition may be placed and suitably aliquoted. Alternatively, the detection reagent and the label may be prepared in a single container means, and in most cases, the kit will also typically include a means for containing the vial(s) in close confinement for commercial sale and/or convenient packaging and delivery.

4.8 Exemplary Definitions

In accordance with the present invention, nucleic acid sequences include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from native sources, chemically synthesized, modified, or otherwise prepared in whole or in part by the hand of man.

Unless defined otherwise, all technical and scientific terms used herein have the same meaning as commonly understood by one of ordinary skill in the art to which this invention belongs. Although any methods and compositions similar or equivalent to those described herein can be used in the practice or testing of the present invention, the preferred methods and compositions are described herein. For purposes of the present invention, the following terms are defined below:

A, an: In accordance with long standing patent law convention, the words “a” and “an” when used in this application, including the claims, denotes “one or more”.

Expression: The combination of intracellular processes, including transcription and translation undergone by a polynucleotide such as a structural gene to synthesize the encoded peptide or polypeptide.

Promoter: a term used to generally describe the region or regions of a nucleic acid sequence that regulates transcription.

Regulatory Element: a term used to generally describe the region or regions of a nucleic acid sequence that regulates transcription.

Structural gene: A gene or sequence region that is expressed to produce an encoded peptide or polypeptide.

Transformation: A process of introducing an exogenous polynucleotide sequence (e.g., a vector, a recombinant DNA or RNA molecule) into a host cell or protoplast in which that exogenous nucleic acid segment is incorporated into at least a first chromosome or is capable of autonomous replication within the transformed host cell. Transfection, electroporation, and naked nucleic acid uptake all represent examples of techniques used to transform a host cell with one or more polynucleotides.

Transformed cell: A host cell whose nucleic acid complement has been altered by the introduction of one or more exogenous polynucleotides into that cell.

Transgenic cell: Any cell derived or regenerated from a transformed cell or derived from a transgenic cell, or from the progeny or offspring of any generation of such a transformed host cell.

Transgenic animal: An animal or a progeny or an offspring of any generation thereof that is derived from a transformed animal cell, wherein the animal's DNA contains an introduced exogenous nucleic acid molecule not originally present in a native, wild type, non-transgenic animal of the same species. The terms “transgenic animal” and “transformed animal” have sometimes been used in the art as synonymous terms to define an animal, the genetic contents of which has been modified to contain one or more exogenous nucleic acid segments.

Vector: A nucleic acid molecule, typically comprised of DNA, capable of replication in a host cell and/or to which another nucleic acid segment can be operatively linked so as to bring about replication of the attached segment. A plasmid, cosmid, or a virus is an exemplary vector.

The terms “substantially corresponds to”, “substantially homologous”, or “substantial identity” as used herein denotes a characteristic of a nucleic acid or an amino acid sequence, wherein a selected nucleic acid or amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared. The percentage of sequence identity may be calculated over the entire length of the sequences to be compared, or may be calculated by excluding small deletions or additions which total less than about 25 percent or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides. Desirably, which highly homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988).

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally occurring animals.

As used herein, a “heterologous” is defined in relation to a predetermined referenced gene sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

As used herein, a “transcription factor recognition site” and a “transcription factor binding site” refer to a polynucleotide sequence(s) or sequence motif(s) which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted on the basis of known consensus sequence motifs, or by other methods known to those of skill in the art.

As used herein, the term “operably linked” refers to a linkage of two or more polynucleotides or two or more nucleic acid sequences in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis sequences that are necessary for efficient transcription and translation (e.g., polyadenylation site(s), mRNA stability controlling sequence(s), etc.

### 5.0 EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### 5.1 Example 1

Growth Arrest of Cells in Quiescent State by Serum Starvation Results in Activation of a Krsl-Related 33-kDa Cytoplasmic Protein Kinase (p33^{QIK}, a Quiescence Induced Kinase)

Activation of p33^{QIK} is induced in harmony with growth arrest of cells in G0/G1 phase by serum starvation or treatment with growth inhibitors. Deactivation of p33^{QIK} closely correlates with activation of p42/44^{Erk} which is an index for induction of the immediate early G1 signaling pathway upon entry of cells into G1 phase. The kinase activity of p33^{QIK} remains inactive throughout S, G2, and M phases until cells are arrested again in quiescent state. Blockage of macromolecular synthesis prevents p33^{QIK} and p42/44^{Erk} from deactivation during G0/G1 transition. Newly synthesized regulators, such as protein phosphatases, are required for deactivation of p33^{QIK} in cells entering into G1 phase. UV irradiation or osmotic shock induces profound elevation of p33^{QIK} activity in quiescent cultures that correlates with induction of apoptosis, but does not induce p33^{QIK} activity in cultures in other phases of the cell cycle. These results illustrate the relevance of p33^{QIK} activity for cell quiescence and apoptosis that may provide an important insight into the signaling cascades for cells arrested in quiescent state and quiescent cells entering into apoptotic phase.

5.1.1 Experimental Procedures

5.1.1.1 Cell Culture and Reagents

Mouse NIH3T3 fibroblast cultures were maintained in Dulbecco's modified Eagle's medium (DMEM)(BRL/GIBCO, Grand Island, N.Y.) supplemented with 5% calf serum, 50 units/ml penicillin, and 5 mcg/ml streptomycin, and cultivated at 37° C.

Mouse 10T1/2 fibroblast cultures were maintained in Basal medium Eagle (BRL/GIBCO, Grand Island, N.Y.) supplemented with 10% heat-inactivated fetal bovine serum, 50 U/ml penicillin, and 5 μg/ml streptomycin, and cultivated at 37° C. Cells synchronized in quiescent state were growth-arrested by serum starvation for 48 hr. Actinomycin D (ActD), cycloheximide (CHXM), 12-0-tetradecanoylphorbol 13-acetate (TPA), staurosporine (STSP) (Sigma Chemical, St. Louis, Mo.), sodium fluoride (NaF), sodium orthovanadate (Na₃VO₄) (Fisher Scientific, Pittsburgh, Pa.), and FR901228 (FR)(NCI, Frederick, Md.) were diluted in cultured medium before treatment of cultured cells. A broad range caspase inhibitor Z-Asp-[(2,6-dichlorobenzoyl)oxy]methane (Z-D-CH₂-DCB) (Dolle et al., 1994; Mashima et al., 1995)(CALBIOCHEM, La Jolla, Calif.) was dissolved in dimethyl sulfoxide and diluted in cultured medium for treatment. Recombinant human Caspase-3 was purchased from Alexis Biochemicals (San Diego, Calif.). Protein phosphatase 1 (PP1) and Lambda protein phosphatase (λPPase) were purchased from NE BioLabs (Beverly, Mass.). Production of rabbit polyclonal antibody Ab-KQ to p63^{Krsl} and p33^{QIK} was made using peptide sequences of Krsl as antigens (Alpha Diagnostic International, San Antonio, Tex.). UV irradiation was carried out using a UV Crosslinker (Stratagene, La Jolla, Calif.).

5.1.1.2 Preparation of Cytosolic Proteins

Cells were lysed with 30 strokes of a tight-fitting Dounce homogenizer in lysis A buffer (10 mM KH₂PO₄, 1 mM EDTA, 5 mM EGTA, 10 mM MgCl₂, 50 mM μ-glycerolphosphate, 1 mM Na₃VO₄, 2 mM dithiothreitol) (Wang et al., 1992). Cytoplasmic proteins (S20) were isolated from the supernatants after centrifugation of crude lysates at 20,000 ×g for 20 min. Protein concentration in S20 was measured by using the BCA assay (Pierce, Rockford, Ill.). Immunoprecipitation of p33^{QIK} was carried out by incubation of S20 with the specific antibody Ab-KQ at 0° C. for 1 hr. Immune complexes were adsorbed to Pansorbin (Staphylococcus aureus) (CALBIOCHEM, La Jolla, Calif.) at 0° C. for 30 min and washed with STE buffer (50 mM Tris-HCl, pH 7.0, 150 mM NaCl, 1 mM Na₂ EDTA) supplemented with 0.1 mM DTT and 0.1% NP-40 and ST buffer supplemented with 1 mM DTT.

5.1.1.3 In-gel Kinase Assay

MBP (BRL/GIBCO, Grand Island, N.Y.) was used as a substrate in the assay and the in-gel kinase assay was performed as previously described (Wang et al., 1992). Briefly, 10% SDS-polyacrylamide gel (SDS-PAG) was copolymerized with 0.4 mg/ml MBP. Cellular proteins were resolved in the MBP-immobilized SDS-PAG, followed by rinsing the gel with 20% isopropanol in buffer B (100 mM Tris, pH 8, 5 mM β-mercaptoethanol). The gel was denatured in buffer B supplemented with 6 M guanidine-HCl and renatured with buffer B supplemented with 0.04% Tween 40 (Kameshita and Fujisawa, 1989). Kinase reaction was carried out by incubating the gel with kinase buffer (20 Mm Tris, pH 7.2, 10 mM MgCl₂, 15 mM β-glycerolphosphate) supplemented with 50 μM γATP and 50 μCi of [γ-³²P]ATP at 22° C. for 30 min. The gel was washed with 1% sodium pyrophosphate in 5% trichloroacetic acid. Protein kinases which phosphorylated MBP in the gel were detected by autoradiography of the [γ³²P]-labeled MBP.

5.1.1.4 Western Immunobloting

Proteins were resolved by electrophoresis in a 10% SDS-PAG and transferred to a nitrocellulose filter sheet (pore size: 0.4μ) (BRL/IGIBCO, Grand Island, N.Y.). Nonspecific protein binding sites in the filter sheet were saturated by incubation of the sheet with 3% non-fat milk in ST buffer (10 mM Tris-HCl, pH 7.2, 150 mM NaCl) at ambient temperature for 30 min. Filters were then incubated with the specific primary antibody at 4° C. for 15 hr. The filters were rinsed three times and incubated with horseradish peroxidase-conjugated antibodies at ambient temperature for 30 min. Antigen-antibody complexes on filters were detected by the Supersignal chemiluminescence kit as indicated by manufacturer (Pierce, Rockford, Ill.) and visualized by autoradiography.

5.1.1.5 Flow Cytometric Analysis

After rinsing with phosphate buffered saline (PBS), cells were trypsinized from culture dishes, rinsed with Ca⁺⁺ and Mg⁺⁺ free PBS, fixed in 70% cold ethanol, and stained with 10 μg/ml propidium iodide (PI) for 30 min (Darzynkiewicz et al., 1995). Flow cytometry analysis was performed on the Coulter EPICS Elite Cytometer (Hialeah, Fla.) using 15 mW air-cooled argon laser producing 488-nm light in which PI fluorescence light emission is collected with a 610LPDC filter. Extended analysis of DNA content and calculation of the percentage of cells in each phase of the cell cycle were performed on Multicycle software (Phoenix Flow System, San Diego, Calif.).

5.1.2 Results

5.1.2.1 Regulation of P33^{QIK} Activity in the Cell Cycle

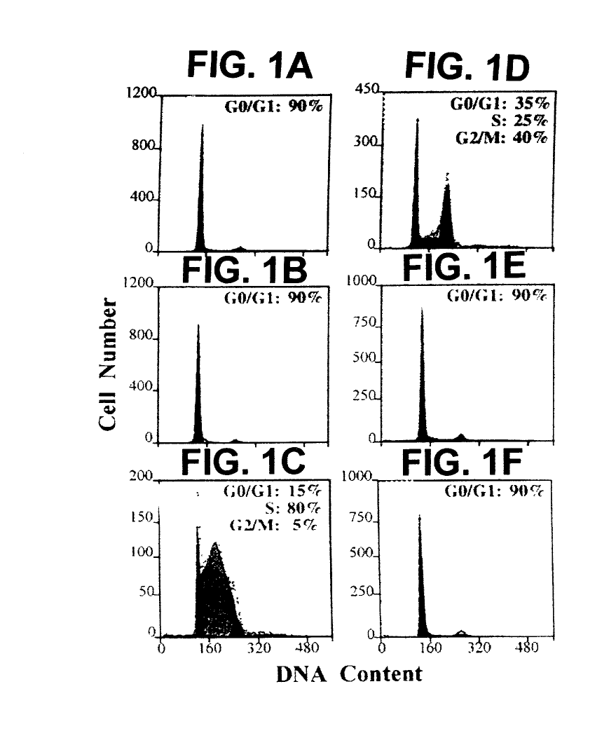

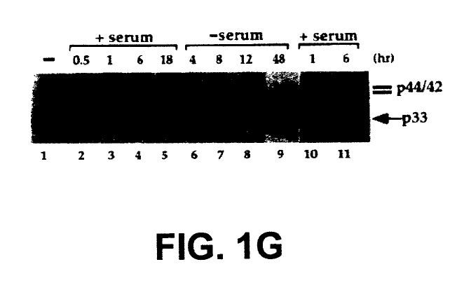

By the in-gel kinase assay using MBP as a substrate, a cytoplasmic protein kinase was detected with molecular mass of 33 kDa, p33^{QIK} in different types of cells, including human epithelial cells (Rajgolikar et al., 1998) and mouse fibroblasts. The kinase activity of p33^{QIK} was closely up-regulated in mouse fibroblasts NIH3T3 and in 10T1/2 cells in the quiescent state which had been induced by serum starvation and down-regulated in cells entering the cell cycle of mitosis. Investigating the regulation of p33^{QIK} activity in the cell cycle, NIH3T3 cultures were growth-arrested and synchronized in quiescent state by serum starvation, and the synchronized quiescent cells were stimulated with serum to enter the cell cycle. After 48 hr of serum starvation, 90% of cells in the NIH3T3 culture remained in G0/G1 phase (FIG. 1A); concomitantly, induction of the kinase activity of p33^{QIK} was detected (FIG. 1G, lane 1). In one hr of serum stimulation of quiescent cultures, transient induction of p44/42^{Erk} activity, an index of the mitogen-activated signaling pathway, was detected (FIG. 1G, lanes 2 and 3) as normally happens during the G0/G1 transition. After 6 hr of serum stimulation, p33^{QIK} activity decreased to a basal level (FIG. 1G, lane 4) while a major population of cells was still in G1 phase (FIG. 1B). After 18 hr of serum treatment, a major population of cells entered the S phase (FIG. 1C) and p33^{QIK} activity remained inactive (FIG. 1G, lane 5). Cultures were then deprived of serum for a second time to arrest cells for 48 hr (FIG. 1G, lanes 6 to 9). Between 4 and 8 hr after initiation of the second serum starvation, 22 and 26 hr after the first entry of cells into the cell cycle, mitosis was observed microscopically. Also, between these two time points, a major population of cells was detected in G2/M phase by flow cytometry (FIG. 1D) and p33^{QIK} activity was gradually induced toward the end of mitosis (FIG. 1G, lane 7). After 12 hr of the second serum starvation when cells were again arrested in G0 state (FIG. 1E), p33^{QIK} activity was again induced (FIG. 1G, lane 8) and remained active in quiescent cultures (FIG. 1G, lane 9) following the extended serum starvation for a total of 48 hr (FIG. 1F). Serum stimulation of these secondly arrested quiescent cultures consistently induced a transient activation of p44/42^{Erk} prior to deactivation of p33^{QIK} again (FIG. 1G, lanes 10 and 11). Clearly, induction of the kinase activity of p33^{QIK} in cells was closely correlated to growth arrest in quiescent state whereas deactivation of p33^{QIK} was correlated with entry of cells into the cell cycle and induction of p44/42^{Erk} activity. By subcellular fractionation, p33^{QIK} activity is mainly detected in the cytoplasmic compartment of quiescent cells and cells entering into G1 phase of the cell cycle.

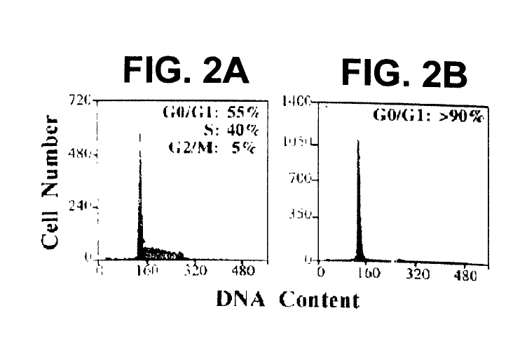

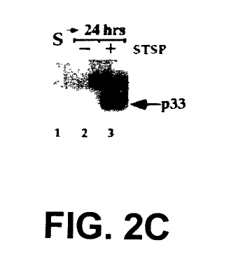

To further determine the correlation between induction of p33^{QIK} activity and growth arrest of cells in G0/G1 phase of the cell cycle, STSP was used to induce growth arrest of cells in G0/G1 phase (Gadbois et al., 1992). STSP has been shown to arrest cells in an early G1 or G2 phase at low (2-20 nM) and high (100-150 nM) concentrations, respectively (Gadbois et al., 1992). STSP is also a potent apoptosis inducing agent (Bertrand et al., 1994) and acts as an inhibitor of PKA, PKC, PKG, S6K, and pp60^{src} (Tamaoki et al, 1986; Kase et al., 1987; Fujita-Yamaguchi and Kathuria, 1988). To reduce the effect of STSP on induction of apoptosis, a low (10 nM) concentration of STSP was used and limited the incubation period of cells with STSP. To generate an adequate culture condition for STSP to growth-arrest cells in G0/G1 phase, NIH3T3 cultures were synchronized in G0 state by serum starvation and released to enter the cell cycle by serum stimulation. After serum stimulation for 18 hr at which time a major population of cells was in S phase (FIG. 1C), STSP was added into the cultures to arrest cell growth. After 24 hr, activation of p33^{QIK} was detected in the STSP-treated culture in which a major population of cells was arrested in G0/G1 state (FIG. 2B; FIG. 2C, lane 3), but was not detected in the untreated culture that exhibited a profile of growing cultures (FIG. 2A FIG. 2C, lane 2).

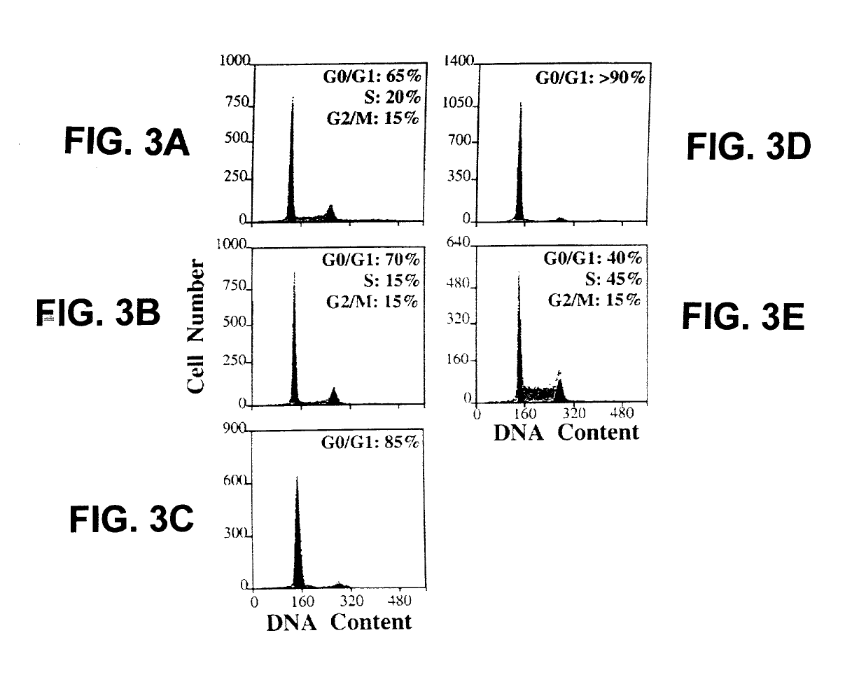

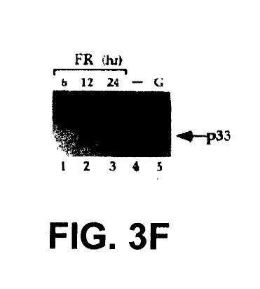

Previously, it was shown that human breast cancer MCF-7 and MDA-MB361 cells treated with an anticancer agent FR resulted in induction of a 33 kDa-MBP kinase activity detected by the in-gel kinase assay (Rajgolikar et al., 1998). FR was reported to induce growth arrest of cells in G0/G1 phase of the cell cycle (Ueda et al., 1994a; 1994b; 1994c). To investigate regulation of p33^{QIK} activity in cells growth-arrested by FR, proliferating NIH3T3 cultures were treated with FR. After 24 hr treatment, growth arrest of cells in G0/G1 phase was evident (FIG. 3C). Accordingly, p33^{QIK} activity was induced in the growth-arrested culture (FIG. 3F, lane 3). Activation of p33^{QIK} was also detected in mouse fibroblast 10T1/2 cultures growth-arrested in G0/G1 phase by serum starvation or treatment with FR. The results indicate that induction of p33^{QIK} activity is tightly correlated with growth arrest of cells in G0/G1 phase of the cell cycle.

5.1.2.2 Correlation Between Deactivation of p33^{QIK} and Activation of p44/42^{ERK}

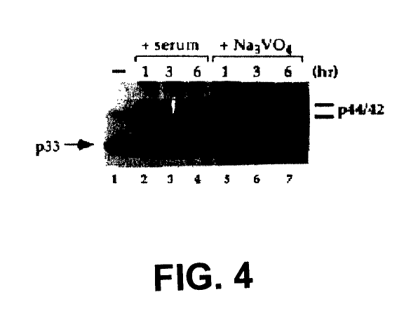

To study deactivation kinetics of p33^{QIK} in cultures by induction of p44/42^{Erk} activity, quiescent NIH3T3 cultures were stimulated to induce p44/42^{Erk} kinase activity with different reagents: serum, TPA, sodium vanadate, okadaic acid, and sodium fluoride. In comparison with serum stimulation of p44/42^{Erk} activity (FIG. 4, lanes 2 to 4), treatment of quiescent NIH3T3 cultures with sodium vanadate, which acts as a general inhibitor of tyrosine phosphatases, resulted in a profound and constant activation of p44/42^{Erk} (FIG. 4, lanes 5 to 7). Concomitantly, an accelerated deactivation of p33^{QIK} was induced in cells treated with sodium vanadate (FIG. 4, lanes 5 to 7) as compared with cells stimulated with serum (FIG. 4, lanes 2 to 4). Cultures stimulated with phorbol ester exhibited similar kinetics to cells stimulated with serum in regulation of p33^{QIK} and p44/42^{Erk} activity; there was a progressive decrease of p33^{QIK} activity and a transient induction of p44/42^{Erk} activity. Consistent with a previous observation (Wang et al., 1992), treatment of cells with okadaic acid activated p63^{Krsl} and treatment of cells with sodium fluoride induced p44/42^{Erk} activity. Also sodium fluoride treatment resulted in deactivation of p33^{QIK}, but okadaic acid treatment did not result in deactivation of p33^{QIK} Down-regulation of p33^{QIK} activity appeared to associate tightly with induction of the immediate early G1 signaling pathway indexed by activation of p42/44^{Erk}.

5.1.2.3 Induction of p33^{QIK} Activity by Inhibitors of Macromolecular Synthesis

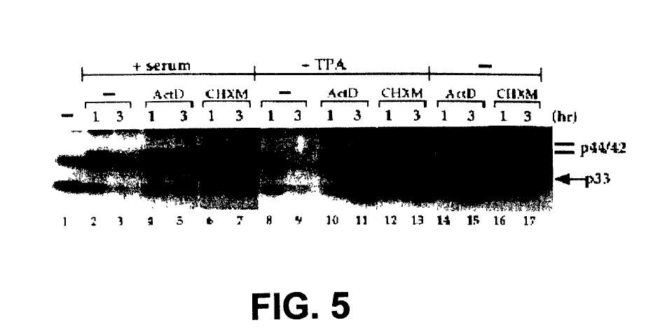

To investigate whether the immediate early G1 gene product is required to down-regulate p33^{QIK} activity, cells were treated with ActD, an inhibitor of gene transcription, or CHXM, an inhibitor of protein synthesis. In the absence of serum, treatment of quiescent cultures with either ActD or CHXM resulted in significant increases of p33^{QIK} activity (FIG. 5, lanes 14 to 17). Co-treatment of cultures with serum and ActD (FIG. 5, lanes 4 and 5) or CHXM (FIG. 5, lanes 6 and 7) blocked the serum-induced down-regulation of p33^{QIK} activity (FIG. 5, lanes 2 and 3). Co-treatment with phorbol ester TPA and ActD (FIG. 5, lanes 10 and 11) or CHXM (FIG. 5, lanes 12 and 13) resulted in not only blockage of the TPA-induced deactivation of p33^{QIK} but also in an increase in the kinase activity of p33^{QIK} (FIG. 5, lanes 10 to 13). In addition, in the presence of ActD or CHXM, treatment of cells with serum or TPA resulted in a constant activation of p44/42^{Erk} (FIG. 5, lanes 4 to 7 and 10 to 13). Treatment with ActD or CHXM appeared to block deactivation of p33^{QIK} and p44/42^{Erk} in cultures after the stimulation with serum or TPA. Activation and deactivation of p44/42^{Erk} is known to result from protein phosphorylation by Mek (Payne et al., 1991; Pelech and Sanghera, 1992; Crews et al., 1992) and dephosphorylation by phosphatase, such as MKP-1 which is an immediate early G1 gene product (Sun et al., 1993; Zheng and Guan, 1993), respectively. Blocking expression of the immediate early G1 gene products by ActD or CHXM appeared to protect p44/42^{Erk} from inactivation. Conceivably, deactivation of p33^{QIK} may also require expression of an immediate early G1 gene product. Additionally, highly activated p33^{QIK} was induced in quiescent cultures by treatment with ActD (FIG. 5, lane 15) or CHXM (FIG. 5, lane 17) for 3 hr while extensive cell death occurred in these cultures. Profound activation of p33^{QIK} appears to be induced in cultures undergoing cell death.

5.1.2.4 Induction of p33^{QIK} Activity Specifically in Quiescent State by Stress Shock

Several groups of investigators reported that activation of a 33 or 36 kDa MBP kinase in cancer cells correlated with induction of apoptosis by anticancer agents or stress shock (Rajgolikar et al., 1998; Lu et al., 1996; Chan et al., 1998; Kakeya et al., 1998; Tang et al., 1998; Walter et al., 1998; Chan et al., 1999). To investigate whether activation of p33^{QIK} was a stress-related event, the kinase activity of p33^{QIK} was measured in cultures entering into different phases of the cell cycle and undergoing stress shock.

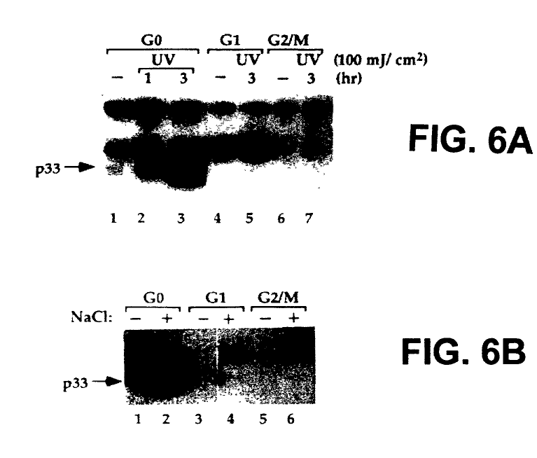

NIH3T3 cultures were growth-arrested in quiescent phase by serum starvation (FIG. 6A and B, lanes 1). Quiescent cultures were released by serum stimulation for 1 or 20 hr, to enter G1 or G2/M phase of the cell cycle, respectively. Cultures were then treated with 100 mJ/cm² of UV irradiation and incubated for another 1 or 3 hr (FIG. 6A-FIG. 6B). The kinase activity of p33^{QIK} was elevated in quiescent cultures by UV irradiation in a time-dependent manner (FIG. 6A, lanes 2 and 3). Increases in activation of p33^{QIK} by UV irradiation were also dose-dependent. In contrast, p33^{QIK} activity was not induced in the cultures in which a major population of cells was in either G1 or G2/M phase by UV irradiation (FIG. 6A, lanes 4 to 7). Similarly, osmotic shock with 300 mM NaCl induced a profound activation of p33^{QIK} in quiescent cultures (FIG. 6B, lane 2), but did not induce p33^{QIK} activity in cultures entering into G1 or G2/M phase (FIG. 6B, lanes 3 to 6). Cultures containing profoundly elevated p33^{QIK} activity exhibited apoptotic morphology with nuclear condensation and cell shrinkage. Addition of serum into cultures immediately after the shock of stress did not suppress apoptotic phenotype or prevent p33^{QIK} from activation. The highly elevated p33^{QIK} activity induced by stress shocks appears to correlate with induction of apoptosis and was a specific event to cells in the quiescent state of the cell cycle.

5.1.2.5 Determination of the Identity of p33^{QIK}

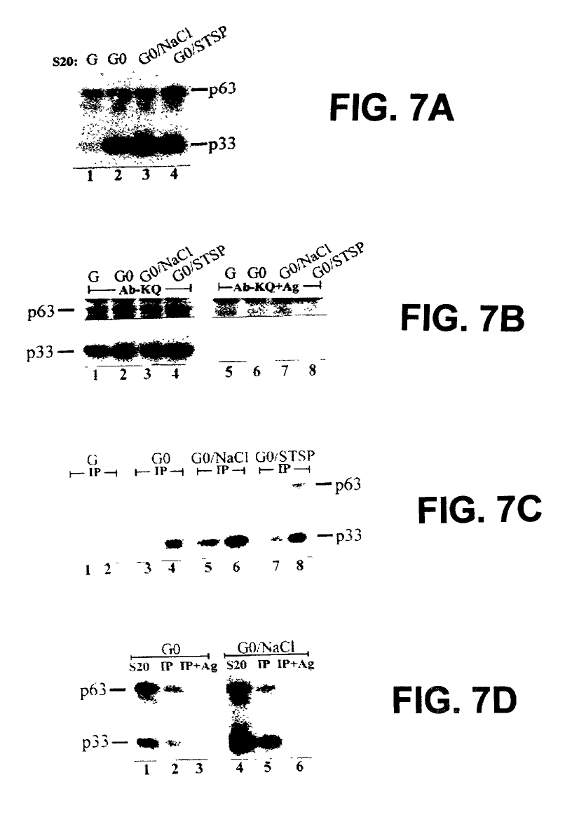

During chromatographic purification of p33^{QIK}, co-elution of p63^{Krsl} and protein kinases with different molecular masses from 55 to 33 kDa was observed. Several groups reported that the proteolysis of Krs/MST proteins by caspases generated a 36 kDa protein kinase (Kakeya et al., 1998; Graves et al., 1998; Watabe et al., 1999). By using peptides derived from Krsl sequences as antigens to generate antibodies, antibodies designated Ab-KQ were obtained that interacted with both p63^{Krsl} and p33^{QIK} in assays of immunoprecipitation and immunoblotting. The Ab-KQ was generated by immunization of rabbits with a Krsl peptide containing residue sequence EIKAKRHDEQQRELEE (SEQ ID NO:3) which has 50% homology with a region of Krs2 (Wang et al., 1992; Creasy and Chemoff, 1995) that is a close family member of Krsl. The peptide shares 20% homologous residues with p21^{Rac}/Cdc42-activated kinase 2 (Pak2) that is a distant family member of Krsl (Martin et al., 1995). To study the specificity of Ab-KQ to p63^{Krsl} and p33^{QIK}, cell lysates were prepared containing activated p63^{Krsl} and p33^{QIK} from STSP-treated quiescent cultures, lysates containing active p33^{QIK} from serum-starved quiescent cultures, and lysates containing profoundly activated p33^{QIK} from osmotic-stressed quiescent cultures. As shown in FIG. 7B, proteins of 63 and 33 kDa were detected in western immunoblotting with Ab-KQ that was completely blocked by preincubation of the antibody with the antigen peptide. The immunoprecipitate obtained with Ab-KQ and tested in the in-gel kinase assay (FIG. 7C) showed increased kinase activities of p63^{Krsl} and p33^{QIK} with increased amounts of Ab-KQ. Ab-KQ did not react with cell lysates containing p63^{Krsl} or p33^{QIK} when Ab-KQ was preincubated with the specific peptide antigen (FIG. 7D). Ab-KQ appeared to interact with p63^{Krsl} and p33^{QIK} equally well.

In comparison, the overall levels of kinase activity of p63^{Krsl} in the cell lysates (FIG. 7A) were in parallel with the protein levels (FIG. 7B). However, the kinase activity of p33^{QIK} (FIG. 7A) was clearly not in parallel with the protein levels (FIG. 7B). In addition, p63^{Krsl} and p33^{QIK} were activated by distinct stimuli. Although QIK was related to Krsl immunologically, the result indicated that p33^{QIK} and p63^{Krsl} were not activated by the same pathway.

5.1.2.6 Regulation of the Kinase Activity of p33^{QIK}

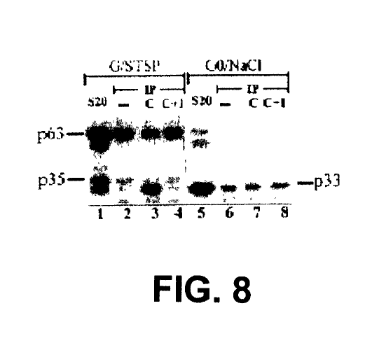

As suggested by other groups, caspase-3 is responsible for proteolytic modification of p63^{Krsl} and generation of a 36-kDa kinase-active peptide (Kakeya et al., 1998; Graves et al., 1998; Watabe et al., 1999). To investigate whether p33^{QIK} activation was derived from p63^{Krsl} , growing or quiescent cultures were treated with STSP or osmotic stress to induce activation of p63 Krsl or p33^{QIK}, respectively. As shown in FIG. 8, p63^{Krsl}, p33^{QIK} and a protein kinase of 35 kDa were detected in the cell lysate and the immune complexes with Ab-KQ prepared from STSP-treated cells. After treatment of the immune complexes with caspase-3, both the kinase activities of p63^{Krsl} and p35 decreased in correlation with an increased p33^{QIK} activity that was completely blocked by a wide range caspase inhibitor Z-D-CH₂-DCB (Dolle et al., 1994; Mashima et al., 1995). However, no change was detected in the kinase activity of p33^{QIK} which was isolated from osmotic-stressed quiescent cultures (FIG. 8, lanes 6 to 8) or growing cultures. Additionally only the STSP-activated p63^{Krsl} and p35 were modified by caspase-3 to generate active p33^{QIK}. In contrast, inactive p63^{Krsl} was not accessible to the caspase-3 modification for activation of p33^{QIK}. Activated p33^{QIK} was also not modified by caspase-3.

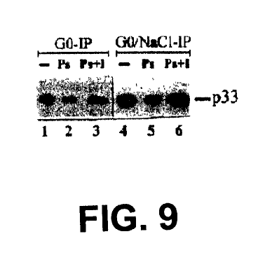

To investigate whether protein phosphorylation was involved in regulation of p33^{QIK} activity, p33^{QIK} was isolated from quiescent cells (FIG. 9, lanes 1 to 3) and quiescent cells treated with osmotic stress (FIG. 9, lanes 4 to 6) by immunoprecipitation with Ab-KQ. p33^{QIK} in the immune complexes was treated with protein phosphatases in the absence or presence of phosphatase inhibitors, a mixture of NaF and Na₃VO4 (Bai et al., 1988). As shown in FIG. 9, treatment of activated p33^{QIK} with protein phosphatase PP1 which can dephosphorylate phosphorylated residues of serine, threonine, tyrosine, and histidine (Gordon, 1991; Zhang et al., 1992; Kim et al., 1993; MacKintosh et al., 1996) resulted in deactivation of p33^{QIK} This deactivation was suppressed by phosphatase inhibitors. A similar result was obtained by using Lambda phosphatase in the reaction. Although dephosphorylation of p33^{QIK} by phosphatases clearly played a role in deactivation of p33^{QIK}, treatment with PP1 did not efficiently deactivate p33^{QIK}, indicating that either PP1 was not the natural regulator for p33^{QIK} or multiple regulators were involved in down-regulation of p33^{QIK} activity.

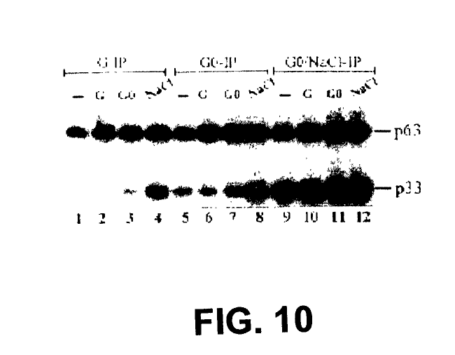

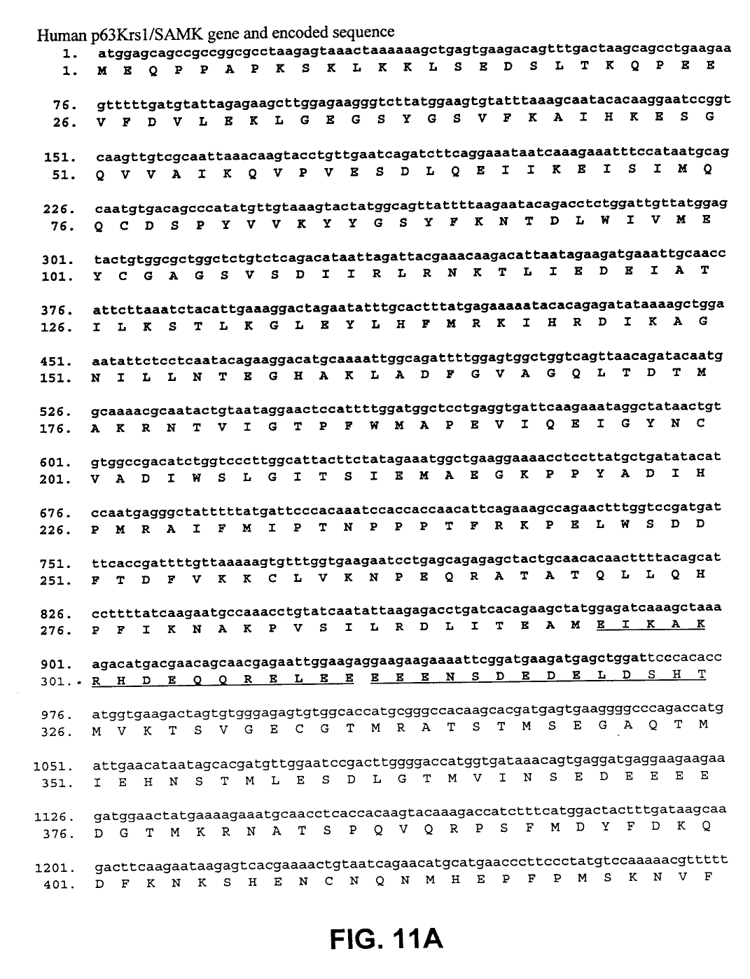

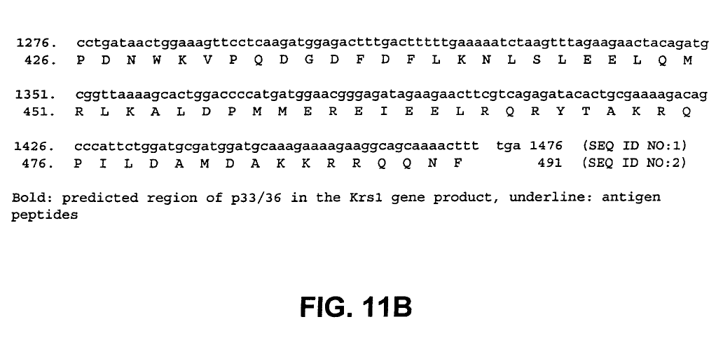

On the other hand, cell lysates prepared from growing, quiescent, or osmotic-stressed quiescent cultures activated p33^{QIK} to different degrees (FIG. 10). Inactive p33^{QIK} in immune complexes prepared from growing cultures was partially activated by the cell lysate prepared from quiescent cells (FIG. 10, lane 3). Both inactive and partially activated p33^{QIK} prepared from growing cultures and quiescent cells, respectively, were fully activated by the cell lysate prepared from osmotic-stressed quiescent cells (FIG. 10, lanes 4 and 8). However, the specific kinase activity of p63^{Krsl} was not changed by incubation with these lysates. Regulation of p33^{QIK} activity apparently was independent from p63^{Krsl} under the circumstance. Distinct activation thresholds of p33^{QIK} appeared to play roles in cell quiescence and apoptosis.

5.1.3 Discussion

The control of shift between G0 and G1 phases is believed to be the main determinant of cell proliferation rate and cell differentiation. Although regulation of signaling pathways and gene expression in cells undergoing different phases of the cell cycle have been heavily studied, the signaling pathway for cells arrested or remained in quiescent state has not been clarified. Studies of p33^{QIK} activity in proliferative and quiescent NIH3T3 cultures demonstrate that induction of the kinase activity of p33^{QIK} closely correlates with cells from serum-starved, STSP, or FR treated cultures arrested in quiescent phase. Deactivation of p33^{QIK} correlates with cell entry into the cell cycle of proliferation. In 1990, a study of expression kinetics of a membrane protein which is encoded by a growth-arrest-specific gene, the gas3 gene was reported (Manfioletti et al., 1990). The gas3 gene mRNA is abundantly expressed in growth-arrested NIH3T3 cells in quiescence by serum starvation for 48 hr. The expression of gas3 mRNA reaches the lowest level 6 hr after serum stimulation and this low level of expression is maintained throughout the cell cycle. The down-regulation of gas3 mRNA has been attributed to the decreased stability of mRNA instead of suppression of transcription (Manfioletti et al., 1990). Consistent with the kinetics of the gas3 gene expression in the cell cycle, p33^{QIK} activity is maximal in cultures growth-arrested in G0 phase by serum starvation, but is reduced to a basal level by 6 hr after serum stimulation and entry into G1 phase. Regulation of the gas3 gene expression and p33^{QIK} activity appears to be modulated by common cellular machinery. Since there is no significant change in expression levels of p33^{QIK} in growing and quiescent cultures, deactivation of p33^{QIK} appears to occur at the posttranslational level by dephosphorylation in contrast to the regulation of gas3 MRNA at the posttranscriptional level. Deactivation of p33^{QIK} closely correlates with activation of p42/44^{Erk} induced during the transition between G0 and G1 phases leading to induction of the immediate early G1 pathway and expression of early G1 gene products (Gille et al., 1992; Blenis, 1993; Whitmarsh et al., 1995). Scholz reported that the proto-oncogene nov mRNA is expressed at high levels in quiescent chicken embryo fibroblasts, but is down-regulated by serum, phorbol ester, or pp60^{v-src} (Scholz et al., 1996). Pretreatment of cells with a PKC inhibitor H7 blocks the down-regulation of nov mRNA, indicating a possible role of PKC in the down-regulation of nov gene expression and supporting the suggestion of the mitogenic signaling pathway in mediating down-regulation of the quiescence-related molecular events (Scholz et al., 1996). Inhibition of macromolecular synthesis by ActD or CHXM in cells entering into G1 phase prevents p33^{QIK} activity from deactivation induced by serum or phorbol ester. Moreover, after induction of p42/44^{Erk} activity by serum or phorbol ester, deactivation of p42/44^{Erk} is also blocked by the treatment of cells with ActD or CHXM. MKP-1, which has been shown to deactivate p42/44Erk by dephosphorylation of p42/44^{Erk} is an immediate early G1 gene product (Sun et al., 1993; Zheng et al., 1993). Consequently, suppression of the immediate early G1 gene expression results in failure of deactivation of p42/44^{Erk}. Conceivably, deactivation of p33^{QIK} may also require a newly synthesized regulator(s) whose expression is upregulated by the immediate early G1 pathway. Since treatment of active p33^{QIK} with protein phosphatases resulted in deactivation of p33^{QIK}, protein phosphorylation/dephosphorylation is involved in activation/deactivation of p33^{QIK}. Newly synthesized phosphatases appear to be required to deactivate p33^{QIK} for cells entering G1 phase of the cell cycle.

Osmotic stress or UV irradiation induced profound increases of p33^{QIK} activity in quiescent cells, but did not induce p33^{QIK} activity in G1 or G2/M phase cells. The profound activation of p33^{QIK} appears to be a quiescence-specific event in response to stress and correlates with exhibition of apoptotic morphology of cell shrinkage, nuclear condensation, loss of cell-cell contact and cytoplasmic blebbing in quiescent cultures after being treated with UV irradiation or shock of osmotic stress. Although the kinase activity of p33^{QIK} is profoundly induced in stressed quiescent cells, change in the protein level is not noted. Distinct thresholds of p33^{QIK} activation appears to play a role in induction of signaling pathways between cell quiescence and apoptosis. Whether protein modification by multiple phosphorylation of different residues is responsible for distinct activation of p33^{QIK} will be determined.

Several groups have reported that a protein kinase of 36 kDa is generated from either Krs/Mst (Graves et al., 1998; Watabe et al., 1999; MacKintosh et al., 1996) or Pak2 (Chan et al, 1998; Tang et al., 1998; Walter et al., 1998; Chan et al., 1999) proteins upon induction of apoptosis by stress or anticancer agents in a caspase-dependent manner. By using Ab-KQ which had been produced from a 16-residue peptide with sequence present in Krsl, it was found that p33^{QIK} and p63^{Krsl} reacted with Ab-KQ, suggesting a homology of same sequences. Since the antigen peptide shares almost no homology with Pak family members, Ab-KQ is unlikely to interact with Pak family members so that p33^{QIK} is probably not related to the Pak family. Although the data shows that p33^{QIK} may be derived from p63^{Krsl} by the proteolytic activity of caspases, only activated p63^{Krsl} is accessible to caspase-3 modification to generate active p33^{QIK} . However, activation of p63^{Krsl} is not a detectable requisite for expression or activation of p33^{QIK} in cells arrested in quiescent state. Cultures were preincubated with inhibitors with broad specificity against various caspases including Z-D-CH₂-DCB (Kakeya et al., 1998) and Z-VAD-FMK (Graves et al., 1998) prior to treatment of cells with serum starvation and shock by osmotic stress. Preincubation of cultures with these caspase inhibitors neither blocked induction of p33^{QIK} activity nor resulted in a changed expression level of p33^{QIK}. Expression and activation of p33^{QIK} appear to be caspase-independent in quiescent cells in contrast to being caspase-dependent in apoptotic cells. Since difference in expression levels of p33^{QIK} is not detectable between growing, quiescent, and apoptotic cultures, regulation of p33^{QIK} expression may not be simple. Regulation of p33^{QIK} expression in cells throughout the cell cycle may be distinct from the apoptosis-related proteolytic generation of p33^{QIK} from activated p63^{Krsl} .

The apoptosis-related process of p33^{QIK} may require more than one caspase to generate intermediate forms of kinase such as active p35 from active p63^{Krsl} and further modification of the active p35 to generate active p33^{QIK}. A similar observation was made by Walter who reported that activation of Pak2 pathway is a multiple process involving proteolysis and protein phosphorylation (Walter et al, 1998). However the p35 kinase is not detected in quiescent cells or quiescent cells undergoing apoptosis. Apparently, different processes are induced to regulate Krsl, p35, and p33 between quiescent and growing cells. Overexpression of a Krsl peptide containing the catalytic domain in cells resulted in induction of apoptosis, activation of JNK signaling pathway, and induction of caspase activity (Graves et al., 1998).

5.2 Example 2

5.2.1 Peptide Sequences Useful in Generating Antibodies of the Present Invention

| | |
|---|---|
| EIKAKRHDEQQRELEE | (SEQ ID NO:3) |
| EEENSDEDELDSHT | (SEQ ID NO:4) |
| PFIKNAKPVSILRDLITEAMEIKAKRHDEQQRELEEEEENSDEDELDSHT | (SEQ ID NO:5) |
| FIKNAKPVSILRDLITEAMEIKAKRHDEQQRELEEEEENSDEDELDSHT | (SEQ ID NO:6) |
| IKNAKPVSILRDLITEAMEIKAKRHDEQQRELEEEEENSDEDELDSHT | (SEQ ID NO:7) |
| KNAKPVSILRDLITEAMEIKAKRHDEQQRELEEEEENSDEDELDSHT | (SEQ ID NO:8) |
| NAKPVSILRDLITEAMEIKAKRHDEQQRELEEEEENSDEDELDSHT | (SEQ ID NO:9) |
| AKPVSILRDLITEAMEIKAKRHDEQQRELEEEEENSDEDELDSHT | (SEQ ID NO:10) |
| KPVSILRDLITEAMEIKAKRHDEQQRELEEEEENSDEDELDSHT | (SEQ ID NO:11) |
| PVSILRDLITEAMEIKAKRHDEQQRELEEEEENSDEDELDSHT | (SEQ ID NO:12) |
| VSILRDLITEAMEIKAKRHDEQQRELEEEEENSDEDELDSHT | (SEQ ID NO:13) |
| SILRDLITEAMEIKAKRHDEQQRELEEEEENSDEDELDSHT | (SEQ ID NO:14) |
| ILRDLITEAMEIKAKRHDEQQRELEEEEENSDEDELDSHT | (SEQ ID NO:15) |
| LRDLITEAMEIKAKRHDEQQRELEEEEENSDEDELDSHT | (SEQ ID NO:16) |
| RDLITEAMEIKAKRHDEQQRELEEEEENSDEDELDSHT | (SEQ ID NO:17) |
| DLITEAMEIKAKRHDEQQRELEEEEENSDEDELDSHT | (SEQ ID NO:18) |
| LITEAMEIKAKRHDEQQRELEEEEENSDEDELDSHT | (SEQ ID NO:19) |
| ITEAMEIKAKRHDEQQRELEEEEENSDEDELDSHT | (SEQ ID NO:20) |
| TEAMEIKAKRHDEQQRELEEEEENSDEDELDSHT | (SEQ ID NO:21) |
| EAMEIKAKRHDEQQRELEEEEENSDEDELDSHT | (SEQ ID NO:22) |
| AMEIKAKRHDEQQRELEEEEENSDEDELDSHT | (SEQ ID NO:23) |
| MEIKAKRHDEQQRELEEEEENSDEDELDSHT | (SEQ ID NO:24) |
| EIKAKRHDEQQRELEEEEENSDEDELDSH | (SEQ ID NO:25) |
| EIKAKRHDEQQRELEEEEENSDEDELDS | (SEQ ID NO:26) |
| EIKAKRHDEQQRELEEEEENSDEDELD | (SEQ ID NO:27) |
| EIKAKRHDEQQRELEEEEENSDEDEL | (SEQ ID NO:28) |
| EIKAKRHDEQQRELEEEEENSDEDE | (SEQ ID NO:29) |
| EIKAKRHDEQQRELEEEEENSDED | (SEQ ID NO:30) |
| EIKAKRHDEQQRELEEEEENSDE | (SEQ ID NO:31) |
| EIKAKRHDEQQRELEEEEENSD | (SEQ ID NO:32) |
| EIKAKRHDEQQRELEEEEENS | (SEQ ID NO:33) |
| EIKAKRHDEQQRELEEEEEN | (SEQ ID NO:34) |
| EIKAKRHDEQQRELEEEEE | (SEQ ID NO:35) |
| EIKAKRHDEQQRELEEEE | (SEQ ID NO:36) |
| IKAKRHDEQQRELEEEEENSDEDELDSHT | (SEQ ID NO:37) |
| KAKRHDEQQRELEEEEENSDEDELDSHT | (SEQ ID NO:38) |
| AKRHDEQQRELEEEEENSDEDELDSHT | (SEQ ID NO:39) |
| KRHDEQQRELEEEEENSDEDELDSHT | (SEQ ID NO:40) |
| RHDEQQRELEEEEENSDEDELDSHT | (SEQ ID NO:41) |
| HDEQQRELEEEEENSDEDELDSHT | (SEQ ID NO:42) |
| DEQQRELEEEEENSDEDELDSHT | (SEQ ID NO:43) |
| EQQRELEEEEENSDEDELDSHT | (SEQ ID NO:44) |
| QQRELEEEEENSDEDELDSHT | (SEQ ID NO:45) |
| QRELEEEEENSDEDELDSHT | (SEQ ID NO:46) |
| RELEEEEENSDEDELDSHT | (SEQ ID NO:47) |
| ELEEEEENSDEDELDSHT | (SEQ ID NO:48) |
| LEEEEENSDEDELDSHT | (SEQ ID NO:49) |
| EEEEENSDEDELDSHT | (SEQ ID NO:50) |
| AMEIKAKRHDEQQRELEEEEENSDEDELDSHTMV | (SEQ ID NO:51) |
| TEAMEIKAKRHDEQQRELEEEEENSDEDELDSHTMVKT | (SEQ ID NO:52) |
| LITEAMEIKAKRHDEQQRELEEEEENSDEDELDSHTMVKTSV | (SEQ ID NO:53) |
| RDLITEAMEIKAKRHDEQQRELEEEEENSDEDELDSHTMVKTSVGE | (SEQ ID NO:54) |
| ILRDLITEAMEIKAKRHDEQQRELEEEEENSDEDELDSHTMVKTSVGECG | (SEQ ID NO:55) |
| VSILRDLITEAMEIKAKRHDEQQRELEEEEENSDEDELDSHTMVKTSVGECGTM | (SEQ ID NO:56) |
| KPVSILRDLITEAMEIKAKRHDEQQRELEEEEENSDEDELDSHTMVKTSVGECGTMRA | (SEQ ID NO:57) |
| EIKAKRHDEQQRELEEEEENSDEDELDSHTM | (SEQ ID NO:58) |
| EIKAKRHDEQQRELEEEEENSDEDELDSHTMV | (SEQ ID NO:59) |
| EIKAKRHDEQQRELEEEEENSDEDELDSHTMVK | (SEQ ID NO:60) |
| EIKAKRHDEQQRELEEEEENSDEDELDSHTMVKT | (SEQ ID NO:61) |
| EIKAKRHDEQQRELEEEEENSDEDELDSHTMVKTS | (SEQ ID NO:62) |
| EIKAKRHDEQQRELEEEEENSDEDELDSHTMVKTSV | (SEQ ID NO:63) |
| EIKAKRHDEQQRELEEEEENSDEDELDSHTMVKTSVG | (SEQ ID NO:64) |
| EIKAKRHDEQQRELEEEEENSDEDELDSHTMVKTSVGE | (SEQ ID NO:65) |
| EIKAKRHDEQQRELEEEEENSDEDELDSHTMVKTSVGEC | (SEQ ID NO:66) |
| EIKAKRHDEQQRELEEEEENSDEDELDSHTMVKTSVGECG | (SEQ ID NO:67) |
| EIKAKRHDEQQRELEEEEENSDEDELDSHTMVKTSVGECGT | (SEQ ID NO:68) |
| EIKAKRHDEQQRELEEEEENSDEDELDSHTMVKTSVGECGTM | (SEQ ID NO:69) |
| EIKAKRHDEQQRELEEEEENSDEDELDSHTMVKTSVGECGTMR | (SEQ ID NO:70) |
| EIKAKRHDEQQRELEEEEENSDEDELDSHTMVKTSVGECGTMRA | (SEQ ID NO:71) |
| EIKAKRHDEQQRELEEEEENSDEDELDSHTMVKTSVGECGTMRAT | (SEQ ID NO:72) |
| EIKAKRHDEQQRELEEEEENSDEDELDSHTMVKTSVGECGTMRATS | (SEQ ID NO:73) |
| EEEENSDEDELDSHTMVKTSVGECGTMRAT | (SEQ ID NO:74) |
| QRELEEEEENSDEDELDSHTMVKTSVGECG | (SEQ ID NO:75) |
| RHDEQQRELEEEEENSDEDELDSHTMVKTS | (SEQ ID NO:76) |

6.0 Deposit of Biological Materials

The subject antibodies, cell lines, hybridoma, and/or cultures of the present invention may be deposited, if necessary, under conditions that assure that access to the cultures will be available during the pendency of this patent application to one determined by the Commissioner of Patents and Trademarks to be entitled thereto under 37 C.F.R. §1.14 and 35 U.S.C. §122. Any such deposits will then be available as required by foreign patent laws in countries wherein counterparts of the subject application, or its progeny, are filed. However, it should be understood that the availability of a deposit does not constitute a license to practice the subject invention in derogation of patent rights granted by governmental action.

Further, any deposits made in conjunction with the present invention may be stored and made available to the public in accord with the provisions of the Budapest Treaty for the Deposit of Biologcal Materials, i.e., they will be stored with all the care necessary to keep them viable and uncontaminated for a period of at least five years after the most recent request for the finishing of a sample of the deposit, and in any case, for a period of at least 30 (thirty) years after the date of deposit or for the enforceable life of any patent which may issue disclosing the cultures. The depositor acknowledges the duty to replace the deposits should the depository be unable to furnish a sample when requested, due to the condition of the deposits. All restrictions on the availability to the public of the subject culture deposits will be irrevocably removed upon the granting of a patent disclosing them. Cell lines, antibodies and hybridomas may, if required, be deposited in the permanent patent collection of the American Type Culture Collection (ATCC), 10801 Uniersity Blvd., Manassas, Va., 20110-2209, USA under the terms of the Budapest Treaty.

5 7.0 References

The following literature citations as well as those cited above are incorporated in pertinent part by reference herein for the reasons cited in the above text: U.S. Pat. No. 4,196,265, issued Apr. 1, 1980.

American Type Culture Collection, Catalogue of Cell Lines and Hybridomas, 7th ed., 1992.

Avrameas, “Natural autoantibodies: Self-recognition and physiological autoimmunity,” In: Natural autoantibodies: Their Physiological Role and Regulatory Significance, Shoenfeld and Isenberg (eds.), CRC Press, Boca Raton, Fla., pp. 1-14, 1993.

Azuma et al., “Correlation Between Augmented Resistance to Influenza Virus Infection and Histological Changes in Lung of Mice Treated with Trehalose-6,6′-dimycolate,” Journal of Biological Response Modifers, 7:473-82, 1988.

Bai, Zhang, Amin, Deans-Zirattu and Lee, FASEB J., 2:3010-3016, 1988.

Bajorin et al., Proc. Annu. Meet. Am. Soc. Clin. Oncol., 7:A967, 1988.

Baker et al., “Ability of Monophosphoryl Lipid A To Augment the Antibody Response of Young Mice,” Infection and Immunity, 56:3064-66, 1988a.

Baker et al., “Enrichment of Suppressor T Cells by Means of Binding to Monophosphoryl Lipid A,” Infection and Immunity, 58:726-31, 1990.

Baker et al., “Inactivation of Suppressor T-Cell Activity by Nontoxic Monophosphoryl Lipid A,” Infection and Immunity, 56:1076-83, 1988b.

Baker et al., “Molecular structures that influence the immunomodulatory properties of the lipid A and inner core region oligosaccharides of bacterial lipopolysaccharides,” Infection Immunity, 62:2257-69, 1994.

Baker et al., “Structural Features That Influence the Ability of Lipid A and Its Analogs To Abolish Expression of Suppressor T Cell Activity,” Infection and Immunity, 60:2694-701, 1992.

Bakker et al., “Melanocyte lineage-specific antigen gp100 is recognized by melanoma-derived tumor-infiltrating lymphocytes,” J. Exp. Med., 179:1005, 1994.

Banchereau and Steinman, Nature, 392:245-51, 1998.

Banerji et al., “Membrane lipid composition modulates the binding specificity of a monoclonal antibody against liposomes,” Biochim. Biophys. Acta., 689:319-26, 1982.

Barnd et al., “Specific tumor histocompatibility complex-unrestricted recognition of tumor-associated mucins by human cytotoxic T cells,” Proc. Natl. Acad. Sci. USA, 86:7159, 1989.

Barnoud et al., Am. J. Surg. Pathol., 24:830-36, 2000).

Bartlett and Zbar, J. Natl. Cancer Inst., 48:1709, 1972.

Bast et al., “BCG and Cancer,” N. Engl. J. Med., 290:1413-20, 1974.

Bennett et al., “Endogenous Production of Cytotoxic Factors in Serum of BCG-Primed Mice by Monophosphoryl Lipid A, a Detoxified Form of Endotoxin,” Journal of Biological Response Modifiers, 7:65-76, 1988

Berkner, Biotechniques, 6:616-27, 1988.

Berra et al., Int. J. Cancer, 36:363-66, 1985.

Berra et al., J. Neurochem., 40:777-82, 1983.

Bertrand, Solary, O'Connor, Kohn and Pommier, Exp. Cell Res., 211:314-321, 1994.

Blenis, Proc. Natl. Acad. Sci. USA, 90:5889-5 892, 1993.

Bogoch, “Demonstration of serum precipitin to brain gangliosides,” Nature, 183:392-93, 1960.

Bouchon et al., Biochem. Internatl., 10:531-38, 1985.

Bowen-Pope et al., Proc. Nat'l Acad. Sci. USA, 81:2396-400, 1984.

Bowness et al., “Clostridiumn perfringens enterotoxin is a superantigen reactive with human T cell receptors V beta 6.9 and V beta 22,” J Exp. Med., 176:893-96, 1992.

Brade et al., “An Artificial Glycoconjugate Containing the Bisphosphorylated Glucosamine Disaccharide Backbone of Lipid A Binds Lipid A Monoclonal Antibodies,” Infection and Immunity, 61:4514-17, 1993.

Brichard et al., “The tyrosinase gene codes for an antigen recognized by autologous cytolytic T lymphocytes on HLA-A2 melanomas,” J. Exp. Med., 178:489, 1993.

Brodin et al., “Mouse monoclonal antibodies with specificity for the melanoma-associated ganglioside disialyllactosyl ceramide (GD3) also react with the structural analogue disialylparagloboside,” Biochim. Biophys. Acta., 837:349-53, 1985.

Brooks et al., Clin. Exp. Immunol., 39: 477, 1980.

Brown et al., J. Biol. Chem., 255:4980-83, 1980.

Burchell et al., J. Immunol., 131:508-13, 1983.

Bystryn et al., Cancer, 61:1065, 1988.

Cahan et al., “Identification of a human neuroectodermal tumor antigen (OFA-I-2) as ganglioside GD2,” Proc. Natl. Acad. Sci. USA, 79:7629-33, 1982.

Campbell et al., Int. J. Cancer, 78:182-88, 1998.

Campbell et al., “Intercellular adhesion molecule-1 expression by bladder cancer cells: functional effects,” J. Urol., 151:1385-90, 1994.

Campbell, in Monoclonal Antibody Technology Laboratory Techniques in Biochemistry and Molecular Biology, Vol. 13, Burden and Von Knippenberg (eds.), Amsterdam, Elseview, pp. 75-83, 1984.

Campbell, In: Monoclonal Antibodly Technology, Laboratory Techniques in Biochemistry and Molecular Biology, Burden and Von Knippenberg, Eds., Vol. 13, pp. 75-83, Elsevier, Amsterdam, pp. 75-83, 1984.

Campisi, Morreo, and Pardee, Exp. Cell Res., 152:459-466, 1984.

Carr and Morrison, “A two-step mechanism for the interaction of Re lipopolysaccharide with erythrocyte membranes,” Rev. Infect. Dis. 6:497-508, 1984.

Carubia et al., Biochem. Biophys. Res. Commun., 120:500-04, 1984.

Chan, Yu and Yang, J. Cell. Physiol., 178:397-408, 1999.

Chan, Yu and Yang, J. Protein Chem., 17:485-494, 1998.

Chang et al., Crit. Rev. Oncol. Hematol., 22:213, 1996.

Chase et al., “Effect of Monophosphoryl Lipid A on Host Resistance to Bacterial Infection,” Infection and Immunity, 53(3):711-12, 1986.

Chen et al., “Activation of Macrophages From Aging Mice by Detoxified Lipid A,” Journal of Leukocyte Biology, 49:416-22, 1991.

Chen etal., Cancer Res., 54:1065-70, 1994.

Cheng et al., “Bacillus Calmette-Gerin interacts with the carboxyl-terminal heparin bindings domain of fibronectin: implications for BCG-mediated antitumor activity,” J. Urol., 152:1275-80, 1994.

Cheresh and Klier, “Disialoganglioside GD3 distributes preferentially into substrate associated microprocesses on human melanoma cells during their attachment to fibronectin,” J. Cell. Biol., 102:1887-97, 1986.

Cheresh et al., “A monoclonal antibody recognizes an 0-acetyl sialic acid in a human melanoma-associated ganglioside,” J. Biol. Chem., 259:7453-59, 1984.

Cheresh et al., “Disialoganglioside GD3 on human melanoma serves as a relevant target antigen for monoclonal antibody-mediated tumor cytolysis,” Proc. Natl. Acad. Sci. USA, 82:5155-59, 1985.

Cheresh et al., “Disialogangliosides GD2 and GD3 are involved in the attachment of human melanoma and neuroblastoma cells to extracellular matrix proteins,” J. Cell. Biol., 102:688-96,1986.

Cheresh et al., “Localization of gangliosides GD2 and GD3 in adhesion plaques and on the surface of human melanoma cells,” Proc. Natl. Acad. Sci. USA, 81:5767-71, 1984.

Cheung et al., “Detection of neuroblastoma cells in bone marrow using GD2 specific monoclonal antibodies,” J. Clin. Oncol., 4:363-69, 1986.

Chu and Sharom, “Gangliosides inhibit T-lymphocyte proliferation by preventing the interaction of interleukin-2 with its cell surface receptors,” Immunology, 79:10-16, 1993.

Ciccarelli, Philipson and Sorrentino, Mol. Cell Biol., 10:1525-1529, 1990.

Coccia, Cicala, Charlesworth, Ciccarelli, Rossi, Philipson and Sorrentino, Mol. Cell Biol., 12:3514-3521, 1992.

Cohen, Science, 259:1691-92, 1993.

Colcher et al., Proc. Natl. Acad. Sci. USA, 78:3199, 1987.

Coligan et al., in Current Protocols in Immunology, Vol. 1, Wiley Interscience. Greene (ed.), 1998.

Coombes et al., Vaccine, 14:1429-38, 1996.

Coulie et al., “A new gene coding for a differentiation antigen recognized by autologous cytologic T lymphocytes on HLA-A2 melanomas,” J. Exp. Med., 180:35, 1994.

Creasy and Chemoff, J. Biol. Chem., 270:21695-21700, 1995.

Crews, Alessandrini and Erikson, Science, 258:478-480, 1992.

Cross, Roberts and Weintraub, Ann. Rev. Cell Biol., 5:341-396, 1989.

Darzynkiewicz, Robison and Crissman, In: Method in Cell Biology, Flow Cytometry, 2nd Ed., Part A, pp. 32-36, 1995.

Deavin et al., Mol Immunol., 33:145-55, 1996.

DeBruijn et al., Eur. J. Immunol., 21:2963-70, 1991.

Denhardt, Edwards and Parfett, Biochim. Biophy. Acta, 865:83-125, 1986.

Dippold et al., “Immunohistochemical localization of ganglioside GD3 in human malignant melanoma, epithelial tumors and normal tissues,” Cancer Res., 45:3699-705, 1985.

Dippold et al., “Inflammatory response at the tumor site after systemic application of monoclonal anti-GD3-ganglioside antibody to patients with malignant melanoma,” Am. Assoc. Cancer Res., 978:247, 1984.

Dippold et al., Proc. Natl. Acad. Sci. USA, 77:6115, 1980.

Dolle, Hoyer, Prasad, Schmidt, Helaszek, Miller and Ator, J. Med Chem., 37:563-564, 1994.

Dresser and Phillips, in Immunopotentiation, CIBA Foundation Symposium 18, Elsevier, Amsterdam, p.3, 1973.

Dwivedi et al., “Plasma lipid-bound sialic acid alterations in neoplastic diseases,” Experientia, 46:91-94, 1990.

Elder, “Skin Cancer,” Cancer, 75:245-56, 1995.

Elliott et al., “The D-Galactosamine Loaded Mouse and Its Enhanced Sensitivity to Lipopolysaccharide and Monophosphoryl Lipid A: A Role for Superoxide,” J. Immunol., 10:69-74, 1991.

Euhus et al., Cancer Immunol Immunother., 29:247-54, 1989.

Fawwaz et al., Statutory Invention Registration Patent No. H819, application no. 6-6-5,439, 1990.

Fischer, Handb. Lipid Res., 6:123-234, 1990.

Fisher-Hoch et al., Proc. Nat'l Acad. Sci. USA, 86:317-21, 1989.

Fitzgerald, “Syphilis vaccine: up-regulation of immunogenicity by cyclophosphamide, Ribi adjuvant, and indomethacin confers significant protection against challenge infection in rabbits,” Vaccine, 9:265-72, 1991.

Fleischmann, Park and Hassan, “Fibronectin expression on surgical specimens correlated with the response to intravesical bacillus Calmette-Guerin therapy,” J. Urol., 149:268-71, 1993.

Flexner et al., Ann. N.Y Acad. Sci., 569:86-103, 1989.

Flexner et al., Vaccine, 8:17-21, 1990.

Foster et al., Cancer Res., 57:3325-30, 1997.

Fraizer et al., Blood, 86:4704-06, 1995.

Fredman et al., Neurol. Res., 8:123-26, 1986.

Freimer et al., “Gangliosides elicit a T-cell independent antibody response,” J. Autoimmun., 6:281-89, 1993.

Freudenberg et al., “ELISA for antibodies to Lipid A, Lipopolyasscharides and other hydrophobic antigens,” Infection, 17:322-24, 1989.

Fujita-Yamaguchi and Kathuria, Biochem. Biophy. Res. Commun., 157:955-962, 1988.

Gadbois, Crissman, Tobey and Bradbury, Proc. Natl. Acad. Sci. USA, 89:8626-8630, 1992.

Gaiger et al., Blood, 96:1334, 2000

Garg and Subbarao, “Immune Responses of Systemic and Mucosal Lymphoid Organs to Pnu-Immune Vaccine as a Function of Age and the Efficacy of Monophosphoryl Lipid A as an Adjuvant,” Infection and Immunity, 60:2329-36, 1992.

Gaugler et al., “Human gene MAGE-3 codes for an antigen recognized on a human melanoma by autologous cytolytic T lymphocytes,” J. Exp. Med., 179:921, 1994.

Gefter et al., Somatic Cell Genet., 3:231-36, 1977.

Gefter, Margulies, Scharff, “A simple method for polyethylene glycol-promoted hybridization of mouse myeloma cells,” Somat. Cell Genet., 3(2):231-236, 1977.

Gennaro et al., Am. J. Ind. Med., 37:275-82, 2000.

Gillard et al., “Antibodies against ganglioside GT₃ in the sera of patients with type I Diabetes mellitus,” J. Immunol., 142:3826-32, 1989.

Gille, Sharrocks and Shaw, Nature, 358:414-417, 1992.

Gillis, Nature, 268:154-56, 1977.

Glynn, McCoy and Fefer, Cancer Res., 28:434-39, 1968.

Goding, “Monoclonal Antibodies: Principles and Practice,” pp. 60-74. 2nd Edition, Academic Press, Orlando, Fla., pp.60-61; 65-66; 71-74, 1986.

Goding, in Monoclonal Antibodies: Principles and Practice, 2d ed., Orlando, Fla., Academic Press, pp. 60-61, 65-66, 71-74, 1986.

Goff et al., Eur. J. Biochem., 130:553-57, 1983.

Gordon, Methods Enzymol., 201:477-482, 1991.

Grabarek et al., “Endotoxic Lipid A Interaction with Human Platelets,” The Journal of Biological Chemistry, 265:8117-21, 1990.

Graus et al., “Distribution of the ganglioside GD3 in the human nervous system detected by R24 mouse monoclonal antibody,” Brain Res., 324:190-94, 1984.

Graves, Gotoh, Draves, Ambrose, Han, Wright, Chemoff, Clark and Krebs, EMBO. J., 17:2224-2234, 1998.

Guzman et al., Cir. Res., 73:1202-07, 1993b.

Guzman et al., Circulation, 88:2838-48, 1993a.

Hachida et al., Transplant Proc., 22:1663-70, 1990.

Hachida et al., Transplantation, 56:479-82, 1993.

Harada et al., Mol. Urol., 3:357-364, 1999.

Hardings et al., “Effects of pH and polysaccharides on peptide binding to class II major histocompatibility complex molecules,” Proc. Natl. Acad. Sci. USA, 88:2740-44, 1991.

Harel et al., Cancer Res., 50:6311, 1990.

Harlow and Lane, Antibodies: A Laboratory Manual, Cold Spring Harbor Laboratory, 1988.

Harlow and Lane, In: Antibodies: A Laboratory Manual, Cold Spring Harbor Laboratory, Cold Spring Harbor, N.Y., 1988.

Helling et al., “Construction of Immunogenic GD₃-conjugate vaccines,” Ann. N.Y. Acad. Sci., 690:396-97, 1993.

Hellstrom et al., “Strong anti-tumor activities of IgG 3 antibodies to a human melanoma-associated ganglioside,” Proc. Natl. Acad. Sci. USA, 82:1499-1502, 1985.

Hirabayashi et al., “Reactivity of mouse monoclonal antibody M2590 against B16 melanoma cells with chemically synthesized GM3 ganglioside,” Biochim. Biophys. Acta, 875:126-28, 1986.

Hirabayashi et al., “Syngeneic monoclonal antibody against melanoma antigen with interspecies cross-reactivity recognize GM₃, a prominent ganglioside of B16 melanoma,” Biol. Chem., 260:13328-33, 1985.

Hirabayashi et al., Jpn. J. Cancer Res., 78:614-20, 1987.

Hoon et al., “Gangliosides from melanoma immunomodulate response of T-cells to interleukin-2,” Cell Immunol., 4:1111-19, 1988.

Horgan, “Total and lipid-bound sialic acid levels in sera from patients with Cancer,” Clin. Chim. Acta., 118:327-31, 1982.

Horibata and Harris, Exp. Cell. Res., 60:61, 1970.

Houghten et al., “Mouse monoclonal IgG3 antibody detecting GD3 ganglioside: A phase I trial in patients with malignant melanoma,” Proc. Natl. Acad. Sci. USA, 82:1242-46, 1985.

Houghton, “Cancer Antigens: Immune Recognition of Self and Altered Self,” J. Exp. Med., 180:1-4, 1994.

Hraba et al., “The Influence of Monophosphoryl Lipid A (MPL™) on Erythrocyte Autoantibody Formation,” Immunobiol., 189:448-56, 1993.

Hunteret al., Vaccine, 9:250: 1991.

Inoue et al, Blood, 88:2267-78, 1996.

Irie and Morton, “Regression of cutaneous metastatic melanoma by intralesional injection with human monoclonal antibody to ganglioside GD2,” Proc. Natl. Acad. Sci. USA, 83:8694-98, 1986.

Irie and Ravindranath, “Gangliosides as targets for monoclonal antibody therapy of cancer,” in Therapeutic monoclonal antibodies, Borrebaeck and Larrick (eds.), Stockton Press, New York, p. 75-94, 1990.

Irie et al., “Human antibody to OFA-I, a tumor antigen, produced in vitro by Epstein-Barr virus transformed human B-lymphoid cell lines,” Proc. Natl. Acad. Sci. USA, 79:5666-70, 1982.

Irie et al., “Melanoma gangliosides and human monoclonal antibody,” in Human Tumor Antigens and Specific Tumor Therapy, Metzgar and Mitchell (eds.), Alan R. Liss, Inc., New York, pp. 115-126, 1989.

Ishioka et al., “MHC interaction and T cell recognition of carbohydrates and glycopeptides,” J. Immunol., 148:2446-51, 1992.

Jackson et al., “Induction of ICAM 1 expression on bladder tumours by BCG immunotherapy,” J. Clin. Pathol., 47:309-12, 1994.

Johnson and Tomai, “A Study of the Cellular and Molecular Mediators of the Adjuvant Action of a Nontoxic Monophosphoryl Lipid A,” Adv. Exp. Med. Biol., 133:567-79, 1988.

Johnson et al., “Structural Characterization of Monophosphoryl Lipid A Homologs Obtained from Salmonella minnesota Re595 Lipopolysaccharide,” J. Biol. Chem., 265:8108-16, 1990.

Johnson et al., “Characterization of a nontoxic monophosphoryl lipid A,” Rev. Infect. Dis., 9:512-16, 1987.

Johnston and Bystryn, “Effect of Cell Wall Skeleton and Monophosphoryl Lipid A Adjuvant on the Immunogenicity of a Murine B16 Melanoma Vaccine,” Journal of the National Cancer Institute, 83:1240-45, 1991.

Jones et al., J Natl Cancer Inst, 66:249-54, 1981.

Kabat et al., “Sequences of Proteins of Immunological Interest,” 5th ed., Public Health Service, National Institutes of Health, Bethesda, Md., pp 647-669, 1991.

Kakeya et al., “Caspase-mediated activation of a 36-kDa myelin basic protein kinase during anticancer drug-induced apoptosis,” Cancer Res., 58(21):4888-4894, 1998.

Kameshita and Fujisawa, Anal. Biochem., 183:139-143, 1989.

Kase, Iwahashi, Nakanishi, Matsuda, Yamada, Takahashi, Murakata, Sato and Kaneko, Biochem. Biophy. Res. Commun., 142:436-440, 1987.

Kass-Eisler et al., Proc. Nat'l Acad. Sci. USA, 90:11498-502, 1993.

Katopodis et al., “Lipid-associated sialic acid test for the detection of human cancer,” Cancer Res., 42:5270-75, 1982.

Kawaguchi et al., “Characteristic mode of action of gangliosides in selective modulation of CD4 on human T lymphocytes,” Biochem. Biophys. Res. Commun., 158:1050-55, 1989.

Kawakami et al., “Cloning of the gene coding for a shared human melanoma antigen recognized by autologous T cells infiltrating into tumor,” Proc. Natl. Acad. Sci. USA, 91:3515, 1994.

Kawakami, Eliyahu, Sakaguchi, Robbins, Rivoltini, Yannelli, Appella and Rosenberg, “Identification of the immunodominant peptides of the Mart-1 human melanoma antigen recognized by the majority of HLA-A2 restricted tumor infiltrating lymphocytes,” J. Exp. Med., 180:347-52, 1994.

Kensil et al., J. Am. Vet. Med. Assoc., 199:1423, 1991.

Kim, Huang, Cohen and Matthews, J. Biol. Chem., 268:18513-18518, 1993.

Kloppel et al., “Glycolipid-bound sialic acid in serum: Increased levels in mice and humans bearing mammary carcinomas,” Proc. Natl. Acad. Sci. USA, 74:3011-13, 1977.

Kohler and Milstein, “Continuous cultures of fused cells secreting antibody of predefined specificity,” Nature, 256(5517):495-497, 1975.

Kohler and Milstein, “Derivation of specific antibody-producing tissue culture and tumor lines by cell fusion,” Eur. J. Immunol., 6(7):511-519, 1976.

Kohler and Milstein, Eur. J. Immunol., 6:511-19, 1976.

Kohler and Milstein, Nature, 256:495-97, 1975.

Kolls et al., Proc. Nat'l Acad. Sci. USA, 91:215-19, 1994.

Koscielak et al., “Glycolipid antigen and its antibody,” Immunochemistry, 5:441-55, 1968.

Kovach et al., “Lipid IV_{A} Inhibits Synthesis and Release of Tumor Necrosis Factor Induced by Lipopolysaccharide in Human Whole Blood Ex Vivo,” J. Exp. Med, 172:77-84, 1990.

Kwok and Higuchi, Nature, 339:237-38, 1989.

Kyogashima et al., Jpn. J. Cancer Res., 78:1229-32, 1987.

Ladisch et al., “Shedding of GD2 ganglioside by human neuroblastoma,” Int. J. Cancer, 39:73-76, 1987.

Lamm et al., “A randomized trial of intravesical doxorubicin and immunotherapy with bacille Calmette-Guérin for transitional-cell carcinoma of the bladder,” N. Engl. J. Med., 325:1205, 1991.

Larsson, Zetterberg and Engstrom, J. Cell Sci., 75:259-268, 1985.

Lengle et al., “Inhibition of the lectin-induced mitogenic response of thymocytes by glycolipids,” Cancer Res., 39:817-922, 1979.

Liepkalns et al., J. Neurochem., 36:1959-65, 1981.

Livingston et al., “Approaches to augmenting immunogenicity of the ganglioside GM₂ in mice: purified GM₂ is superior to whole cells,” J. Immunol., 138:1524-29, 1987a.

Livingston et al., “The Serologic Response to Meth A Sarcoma Vaccines After Cyclophosphamide Treatment is Additionally Increased by Various Adjuvants,” The Journal of Immunology, 135(2):1505-09, 1985.

Livingston et al., “Vaccines containing purified GM₂ gangliosides elicit GM₂ antibodies in melanoma patients,” Proc. Natl. Acad. Sci. USA, 84:2911-15, 1987b.

Ljunggren et al., Nature, 346:476-80, 1990.

Lozzio and Lozzio, Blood, 45:321-34, 1975.

Lu, Sato, Cao and Richie, Proc. Natl. Acad. Sci. USA, 93:8977-8982, 1996.

MacKintosh, Garton, McDonnell, Barford, Cohen, Tonks and Cohen, FEBS Lett., 397:235-238, 1996.

Madonna and Vogel, “Induction of Early-Phase Endotoxin Tolerance in Athymic (Nude) Mice, B-Cell-Deficient (xid) Mice, and Splenectomized Mice,” Infection and Immunity, 53:707-10, 1986.

Mahvi et al., Immunology and cell Biology, 75:456-60, 1997.

Manfioletti, Brancolini, Avanzi and Schneider, Mol. Cell Biol., 13:4976-4985, 1993.

Manfioletti, Ruaro, Sal, Philipson and Schneider, Mol. Cell Biol., 10:2924-2930, 1990.

Maratea et al., Gene, 40:39-46, 1985.

Martin, Bollag, McCormick and Abo, EMBO J., 14:1970-1978, 1995.

Mashima, Naito, Fujita, Noguchi and Tsuruo, Biochem. Biophys. Res. Commun 21:1185-1192, 1995.

Masihi et al., “Effects of Nontoxic Lipid A and Endotoxin on Resistance of Mice to Toxoplasma gondii,” Journal of Biological Response Modifiers, 7:535-39, 1988

Melling et al., J. Immunol, 117:1267-74, 1976.

Menssen et al., J. Cancer Res. Clin. Oncol., 126:226-32, 2000.

Merrifield, J. Am. Chem. Soc., 85:2149-46, 1963.

Miller and Esselman, “Modulation of immune response by antigen reactive lymphocytes after cultivation with gangliosides,” J. Immunol., 115:839-43, 1975.

Minden, “Shared Antigens Between Animal and Human Tumors and Microorganisms,” in BCG in Cancer Immunotherapy, Lamoureux, Turcotte and Portelance (eds.); pp. 73-81, 1976.

Miotti et al., Cancer Res., 65:826, 1985.

Mitchell et al., “Active specific Immunotherapy of melanoma with allogeneic cell lysates: Rationale, results and possible mechanisms of action,” Ann. N.Y Acad. Sci., 690:153-66, 1993.

Mitchell et al., “Active specific immunotherapy of melanoma: Phase I trial of allogeneic lysates and a novel adjuvant,” Cancer Res., 48:5883-93, 1988.

Mitchell et al., “Active-Specific Immunotherapy for Melanoma,” Journal of Clinical Oncology, 8:856-59, 1990.

Miyake et al., Cancer Res., 48:6154-60, 1988.

Mooney et al., “Bacterial superantigen signaling via HLA class II on human B lymphocytes,” Mol. Immunol., 31:675-81, 1994.

Morrison et al., “Specific ganglioside binding to receptor sites on T lymphocytes that couple to ganglioside-induced decrease of CD4 expression,” Life Sci., 45:1219-24, 1989.

Morton and Ravindranath, in Cancer Medicine, 3rd ed., Holland et al. (eds.), Lea and Febiger, Philadelphia, p.967, 1993.

Morton et al., “Polyvalent Melanoma Vaccine Improves Survival of Patients with Metastatic Melanoma,” John Wayne Cancer Institute at Saint John's Hospital and Health Center, Santa Monica, Calif., reprinted from Specific Immunotherapy of Cancer with Vaccines, Volume 690 of the Annals of the New York Academy of sciences, 1993.

Morton et al., Ann. Surg., 216:463, 1992.

Morton et al., in Biological Function of Gangliosides, Progress in Brain Research, Vol. 101, pp 251-275, 1994.

Mosmann and Coffinan, Ann. Rev. Immunol., 7:145-73, 1989.

Munjal et al., “Combined measurement and significance of lipid-bound sialic acid and carcinoembryonic antigen in detection of human cancer,” Diagn. Immunol., 2:36-43, 1984.

Murphy et al., Proc. Natl. Acad. Sci. USA, 83:8258-62, 1986.

Myers et al., “Monophosphoryl Lipid A Behaves as a T-Cell-Independent Type 1 Carrier for Hapten-Specific Antibody Responses in Mice,” Infection and Immunity, 63:168-74, 1995.

Naiki et al., “Properties of antisera to ganglioside GM₁ and AsialoGM₁”, J. Immunol., 113:84-93, 1974.

Nair et al., Nature Biotechnol., 16:364-69, 1998.

Natoli et al, “A murine monoclonal antibody detecting the ganglioside GM2: Characterization of cell surface reactivity,” Cancer Res., 46:4116-20, 1986.

Nonomura et al., Hinyokika Kiyo, 45:593-97, 1999.

Nudelman et al., “Characterization of a human melanoma-associated ganglioside antigen defmed by a monoclonal antibody 4.2,” J. Biol. Chem., 257:12752-56, 1982.

Odean et al., “Involvement of Gamma Interferon in Antibody Enhancement by Adjuvants,” Infection and Immunity, 58:427-32, 1990.

Oji et al., Jpn. J. Cancer Res., 90:194-204, 1999.

Old et al., Ann. N.Y. Acad. Sci., 101:80-106, 1962.

Pan et al., Leukemia, 14:1634, 2000.

Pardee, Science, 246:603-608, 1989.

Parker et al., J. Immunol., 152:163, 1994.

Pascal et al, “Immunochemical studies on normal and Tay-Sachs' brain gangliosides,” Proc. Soc. Exp. Biol. Med., 121:739-43, 1966.

Patek, Collins and Cohn, “Transformed cell lines susceptible or resistant to in vivo surveillance against tumorigenesis.” Nature, 276:510-11, 1978.

Patmasiriwat et al., Leukemia, 13:891-900, 1999.

Paul, Fundamental Immunology, 3rd ed., Raven Press, pp.243-47, 1993.

Payne, Rossomando, Martino, Erikson, Her, Shabanowitz, Hunt, Weber and Sturgill, EMBO J., 10:885-892, 1991.

Pelech and Sanghera, Science, 257:1355-1356, 1992.

Portoukalian et al., “Humoral immune response in disease-free advanced melanoma patients after vaccination with melanoma-associated gangliosides,” Int. J. Cancer, 49:893-99, 1991.

Portoukalian, “Alteration of gangliosides in plasma and red cells of human bearing melanoma tumors,” Biochem. Biophys. Res. Commun., 85:916-20, 1978.

Portoukalian, “Immunoregulatory activity of gangliosides shed by melanoma tumors,” in Gangliosides and Cancer, Oettgen (ed.), New York, VCH Publishers, pp. 207-16, 1989.

Powell and Newman (eds.), “Vaccine Design (the subunit and adjuvant approach),” Plenum Press, NY, 1995.

Prokazova et al., “Sialylated lactosylceramides. Possible inducers of non-specific immunosuppression and atherosclerotic lesions,” Eur. J. Biochem., 171:1-10, 1988.

Pukel et al., “GD3, a prominent ganglioside of human melanoma: Detection and characterization of mouse monoclonal antibody,” J. Exp. Med., 155:1133-47, 1982.

Qureshi et al., “Purification and structural determination of nontoxic lipid A obtained from the Lipopolysaccharide of Salmonella typhimurium,” J. Biol. Chem., 257:11808-15, 1985.

Rabinovich et al., “Vaccine Technologies: View to the Future,” Science, 265:1401-02, 1994.

Raines and Ross, J. Biol. Chem., 257:5154-60, 1982.

Rajgolikar, Chan and Wang, Breast Cancer Res. Treat., 51:29-38, 1998.

Rapport and Graf, “Immunochemical Reactions of Lipids,” Prog. Allergy, 13:273-331, 1969.

Ravindranath and Irie, in Malignant Melanoma: Biology, Diagnosis, and Therapy, Nathanson (ed.), Kluwer Acad., Boston, p. 17, 1988.

Ravindranath and Morton, “Role of gangliosides in active immunotherapy with melanoma vaccine,” Int. Rev. Immunol., 7:303, 1991.

Ravindranath et al., “An epitope common to gangliosides O-acetyl GD3 and GD3 recognized by antibodies in melanoma patients after active specific immunotherapy,” Cancer Res., 49:3891-97, 1989.

Ravindranath et al., “Attachment of Monophosphoryl Lipid A (MPL) to Cells and Liposomes Augments Antibody Response to membrane-bound Gangliosides,” Journal of Autoimmunity, 7:803-16, 1994b.

Ravindranath et al., “Efficacy of tumor cell vaccine after incorporating monophosphoryl lipid A (MPL) in tumor cell membranes containing tumor-associated ganglioside,” Experientia, 50:648-653, 1994a.

Ravindranath et al., “Factors affecting the fine specificity and sensitivity of serum antiganglioside antibodies in ELISA,” J. Immunol. Methods, 169:257-72, 1994c.

Ravindranath et al., “Ganglioside GM₃:GD₃ Ratio as an Index for the Management of Melanoma,” Cancer, 67:3029-35, 1991.

Ravindranath et al., “Human melanoma antigen 0-acetylated Ganglioside GD₃ is recognized by Cancer antennarius lectin,” J. Biol. Chem., 263:2079-86, 1988.

Real et al., “Class I (unique) tumor antigens of human melanoma. Identification of a 90,000 dalton cell surface glycoprotein by autologous antibody,” J. Exp. Med., 160:1219, 1984.

Reeves et al., Cancer Res., 56:5672-77, 1996.

Reisfeld et al., Melanoma Antigens and Antibodies, p.317, 1982.

Ribi et al., “Lipid A and immunotherapy,” Rev. Infect. Dis., 6:567-72, 1984.

Ribi et al., “Modulation of humoral and cell mediated immune responses by a structurally established nontoxic lipid A,” in Immunobiology and Immunopharmacology of Bacterial Endotoxins, Szentivanji and Friedman (eds.), Plenum Press, New York, pp. 407-420, 1986.

Ribi, “Beneficial modification of the endotoxin molecule,” J. Biol. Resp. Mod., 3:1-9, 1984.

Rickman et al., Lancet 337:998, 1991.

Rolland, Crit. Rev. Therap. Drug Carrier Systems, 15:143-98, 1998.

Rosenberg et al., Ann. Surg., 210:474, 1989.

Rosenberg et al., N. Engl. J. Med., 319:1676, 1988.

Rosenfeld et al., Science, 252:431-34, 1991.

Rothbard and Taylor, EMBO J.., 7:93-100, 1988.

Rott et al., “Protection from experimental allergic encephalomyelitis by application of a bacterial superantigen,” Int. Immunol., 4:347-53, 1992.

Sato et al., Science, 273:352, 1996.

Sato et al., “Cytoplasmic membrane-associated protein (CAP) isolated from Streptococcus pyrogenes: as a new bacterial superantigen,” Microbiol. Immuno., 38:139-47, 1994.

Satoh et al., Pathol. Int., 50:458-71, 2000.

Schneider, King and Philipson, Cell, 54:787-793, 1988.

Scholz, Martinerie, Perbal and Hanafusa, Mol. Cell Biol., 16:481-486, 1996.

Schuster et al., “Production of antibodies against phosphocholine, phosphatidylcholine, sphingomyelin, and lipid A by injection of liposomes containing lipid A,” J. Immunol., 122:900-05, 1979.

Schwab et al., “Superantigen can reactivate bacterial cell wall-induced arthritis,” J. Immunol., 150:4151-59, 1993.

Shafer and Spitznagel, “Sensitivity of Salmonella typhimurium to polymorphonuclear granulocyte extracts: Role of lipid A,” Rev. Infect. Dis., 6:577-81, 1984.

Shepard et al., J. Clin. Immunol., 11: 117-27, 1991.

Sherwin et al., “The production of antisera to gangliosides from human nervous tissue,” Canad. J. Biochem., 42:1640-48, 1964.

Shimizu et al., Int. J. Gynecol. Pathol., 19:158-63, 2000.

Shy et al., “Antibodies to GM₁ and GD_{1b} in patients with motor neuron disease with plasma cell dyscrasia,” Ann. Neurol., 25:511-18, 1989.

Siddiqui et al., Cancer Res., 44:5262-65, 1984.

Sidell et al., Cancer Immunol Immunother, 7:151-55, 1979.

Sigma Cell Culture, Volume 9, Number 2, 1993.

Skeiky et al., Infection and Immun., 67:3998-4007, 1999.

Slavin and Strober, Nature, 272:624-26, 1977.

Spinsanti et al., Leuk. Lymphoma, 38:611-19, 2000.

Stiess and Krüger, “Mammalian Cell Culture Media-Overview and Applications,” The Source (Sigma Cell Culture Technical and Product News), 9:1-8, 1993.

Stoute etal. New Engl. J. Med., 336:86-91, 1997.

Sun, Charles, Lau and Tonks, Cell, 75:487-493, 1993.

Svennerholm et al., “Tumor gangliosides as targets for active specific immunotherapy of melanoma in man,” in Biological Function of Gangliosides, Progress in Brain Research, Vol. 101, 1994.

Tai et al., “Ganglioside GM2 as a human tumor antigen (OFA-I-1),” Proc. Natl. Acad. Sci., 80:5392-96, 1983.

Takada et al., “Molecular and Structural Requirements of a Lipoteichoic Acid from Enterococcus hirae ATCC 9790 for Cytokine-Inducing, Antitumor, and Antigenic Activities,” Infection and Immunity, 63:57-65, 1995.

Takahashi et al., J. Immunol., 140:3244, 1988.

Tamaki et al., Leukemia, 13:393-99, 1999.

Tamaoki, Nomoto, Takahashi, Ikato, Morimoto and Tomita., Biochem. Biophys. Res. Commun., 135:397-402, 1986.

Tamauchi et al., Immunology, 50:605, 1983.

Tanamoto, “Dissociation of Endotoxic Activities in a Chemically Synthesized Lipid A Precursor after Acetylation,” Infection and Immunity, 63:690-92, 1995.

Tanamoto, “Free Hydroxyl Groups Are Not Required for Endotoxic Activity of Lipid A,” Infection and Immunity, 62:1705-09, 1994a.

Tanamoto, FEBS Lett., 351:325-29, 1994b.

Tang, Chang, Chan, Yang, Ni and Yu, J. Cell Biochem., 70:442-454, 1998.

Tautu et al., “Improved procedure for determination of serum lipid-associated sialic acid: Application for early diagnosis of colorectal cancer,” J. Natl. Cancer Inst., 80:1333-37, 1988.

Taylor, Wang and Erikson, Proc. Natl. Acad. Sci. USA, 93:10099-10104, 1996.

Thor et al., Cancer Res., 46:3118, 1986.

Thurin et al., “Proton NMR and fast-atom bombardment mass spectrometry analysis of the melanoma-associated ganglioside 9-0-acetyl GD3,” J Biol. Chem., 260:14556-563, 1985.

Timmerman and Levy, Ann. Rev. Med., 50:507-29, 1999.

Tomai and Johnson, “T Cell and Interferon-□ Involvement in the Adjuvant Action of a Detoxified Endotoxin,” Journal of Biological Response Modifiers, 8:625-43, 1989.

Tomai et al., “The Adjuvant Properties of a Nontoxic Monophosphoryl Lipid A in Hyporesponsive and Aging Mice,” Journal of Biological Response Modifiers, 6:99-107, 1987.

Tsuchida et al., “Gangliosides as tumor markers of human melanoma: bio-chemical and immunologic assays,” in New Horizons of Tumor Immunotherapy, Torisu and Yoshida (eds.), pp. 315-325, 1989.

Tsuchida et al., “Gangliosides of Human Melanoma: Altered Expression in Vivo and in Vitro,” Cancer Research, 47:1278-81, 1987.

Tsuchida et al., J. Dermatol., 11:129-38, 1984.

Tuting et al., J. Immunol., 160:1139-47, 1998.

Ueda, Manda, Matsumoto Mukumoto, Nishigaki, Kawamura and Shimomura, J. Antibiotics, 47:315-323, 1994b.

Ueda, Nakajima, Hori, Fujita, Nishimura, Goto, and Okuhara, J. Antibiotics, 47:301-310, 1994a.

Ueda, Nakajima, Hori, Goto and Okuhara, Biosci. Biotech. Biochem., 58:1579-1583, 1994c.

Ulmer et al., Science, 259:1745-49, 1993.

Vadhan-Raj et al., J Clin. Oncol., 6:1636, 1988.

van der Bruggen et al., “A gene encoding an antigen recognized by cytolytic T lymphocytes on a human melanoma,” Science, 264:716, 1991.

Verma et al., “Adjuvant Effects of Liposomes Containing Lipid A: Enhancement of Liposomal Antigen Presentation and Recruitment of Macrophages,” Infection and Immunity, 60:2438-44, 1992.

Vijayasaradhi et al., “The melanoma antigen gp75 is the human homologue of the mouse b (brown) locus gene product,” J. Exp. Med., 171:1375, 1990.

Von Bultzingslowen, Pneumologie, 53:266-75, 1999.

Vosika et al., Cancer Immunol. Immunother., 18:107, 1984.

Walter, Huang, Jakobi, Tuazon, Alnemri, Litwack and Traugh, J. Biol. Chem., 273:28733-28739, 1998.

Wang and Erikson, Mol. Biol. Cell, 3:1329-1337, 1992.

Watabe, Kakeya and Osada, Oncogene, 18:5211-5220, 1999.

Watson, Ralph, Sarkar and Cohn, “Leukemia viruses associated with mouse myeloma cells,” Proc. Natl. Acad. Sci. USA, 66:344, 1970.

Welcher, Suter, Leon, Snipes and Shooter, Proc. Natl. Acad. Sci. USA, 88:7195-7199, 1991.

Westrick et al., Biochim. Biophys. Acta, 750:141-48, 1983a

Westrick et al., Cancer Res., 43:5890-94, 1983b

Whisler and Yates, “Regulation of lymphocyte responses by human gangliosides. I. Characteristics of inhibitory effects and the induction of impaired activation,” J. Immuunol., 125:2106-12, 1980.

Whitmarsh, Shore, Sharrocks and Davis, Science, 269:403-407, 1995.

Wide et al., in Radioimmunoassay Methods, Kirkham and Hunter (eds.), E. and S. Livingstone, Edinburgh, 1970.

Wilschut, “Preparation and properties of phospholipid vesicles,” in Methodologie des liposomes appliquee a la pharmacologie et a la biologies cellulaire, Leserman and Barbet (eds.), INSERM, Paris, pp. 1-10, 1982.

Yamaguchi et al., “Cell-surface antigens of melanoma recognized by human monoclonal antibodies,” Proc. Natl. Acad. Sci. USA, 84:2416-20, 1987.

Yamamoto et al., “In vitro Augmentation of Natural Killer Cell Activity and Production of Interferon-α/αand -βwith Deoxyribonucleic Acid Fraction from Mycobacterium bovis BCG,” Jpn. J. Cancer Res., 79:866-73, 1988.

Yeh et al., “A cell-surface antigen which is present in the ganglioside fraction and shared by human melanomas,” Int. J. Cancer, 29:269-75, 1982.

Yin et al., “Effect of Various Adjuvants on the Antibody Response of Mice to Pneumococcal Polysaccharides,” Journal of Biological Response Modifiers, 8:190-205, 1989.

Yokoyarna et al., “Immunochemical studies with gangliosides,” J. Immunol., 90:372-80, 1963.

Zapata et al., Protein Eng., 8:1057-62, 1995.

Zhang, Bai, Deans-Zirattu, Browner and Lee, J. Biol. Chem., 267:1484-1490, 1992.

Zheng and Guan, J. Biol. Chem., 268:16116-16119, 1993.

Zitvogel et al., Nat. Med., 4:594-600, 1998.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. Accordingly, the exclusive rights sought to be patented are as described in the claims below.

What is claimed is:

1. An isolated peptide of from 9 to about 70 amino acids in length, said peptide comprising at least a first contiguous amino acid sequence according to any one of SEQ ID NO:3 through SEQ ID NO:76.

2. The isolated peptide of claim 1, of from 9 to about 60 amino acids in length.

3. The isolated peptide of claim 2, of from 9 to about 50 amino acids in length.

4. The isolated peptide of claim 3, of from 9 to about 40 amino acids in length.

5. The isolated peptide of claim 4, of from 9 to about 30 amino acids in length.

6. The isolated peptide of claim 5, of from 9 to about 20 amino acids in length.

7. The isolated peptide of claim 1, said peptide comprising a first contiguous amino acid sequence selected from the group consisting of SEQ ID NO:3 and SEQ ID NO:4.

8. The isolated peptide of claim 1, said peptide consisting essentially of a contiguous amino acid sequence according to any one of SEQ ID NO:3 through SEQ ID NO:76.

9. The isolated peptide of claim 8, said peptide consisting of a contiguous amino acid sequence according to any one of SEQ ID NO:3 through SEQ ID NO:76.

10. The isolated peptide of claim 1, said peptide further comprising at least a second contiguous amino acid sequence according to any one of SEQ ID NO:3 to SEQ ID NO:76.

11. An isolated polypeptide consisting essentially of the amino acid sequence from position 1 to position 322 of SEQ ID NO:2.

12. The isolated polypeptide of claim 11, consisting of the amino acid sequence from position 1 to position 322 of SEQ ID NO:2.

13. A purified antibody, or antigen-binding fragment thereof, that is immunospecific for the peptide of claim 1, the polypeptide of claim 11, or a native, or biologically-active mammalian p33^{QIK} or p63^{Krsl} peptide or polypeptide.

14. The antibody or antigen-binding fragment of claim 13, wherein said antibody or antigen-binding fragment is immunospecific for the peptide of claim 1.

15. The antibody or antigen-binding fragment of claim 13, wherein said antibody or antigen-binding fragment is immunospecific for the polypeptide of claim 11.

16. The antibody or antigen-binding fragment of claim 13, wherein said antibody or antigen-binding fragment is immunospecific for a human p33^{QIK} or p63^{Krsl} peptide or polypeptide.

17. The antibody or antigen-binding fragment of claim 16, wherein said antibody or antigen-binding fragment is immunospecific for a native, or biologically-active human p33^{QIK} or p63^{Krsl} peptide or polypeptide.

18. The antibody or antigen-binding fragment of claim 13, wherein said antibody or antigen-binding fragment is immunospecific for the peptide of claim 8.

19. The antibody or antigen-binding fragment of claim 13, wherein said antibody or antigen-binding fragment is immunospecific for the peptide of claim 9.

20. The antibody or antigen-binding fragment of claim 13, wherein said antibody or antigen-binding fragment is an IgG or an IgM antibody or antigen-binding fragment thereof.

21. The antibody or antigen-binding fragment of claim 20, wherein said antigen-binding fragment is an scfv, Fv, Fab′, Fab or F(ab′)₂ fragment.

22. The antibody or antigen-binding fragment of claim 13, wherein said antibody is a monoclonal antibody.

23. The antibody or antigen-binding fragment of claim 22, wherein said antibody is a murine monoclonal antibody.

24. The antibody or antigen-binding fragment of claim 13, wherein said antibody is produced by the cell line deposited with the American Type Culture Collection as Accession No. X.

25. The antibody or antigen-binding fragment of claim 13, wherein said antibody is comprised within a polyclonal antiserum.

26. The antibody or antigen-binding fragment of claim 25, wherein said antibody is comprised within a polyclonal serum designated Ab-KQ.

27. The antibody or antigen-binding fragment of claim 13, wherein said antibody is a dimeric, trimeric or multimeric antibody.

28. The antibody or antigen-binding fragment of claim 13, wherein said antibody is a human or a humanized antibody.

29. The antibody or antigen-binding fragment of claim 13, wherein said antibody comprises an antigen-binding region operatively attached to a human antibody framework or constant region.

30. The antibody or antigen-binding fragment of claim 13, wherein said antibody is a chimeric antibody.

31. The antibody or antigen-binding fragment of claim 13, wherein said antibody is a recombinant antibody.

32. The peptide of claim 1, the polypeptide of claim 11, or the antibody or antigen-binding fragment of claim 13, further comprising at least a first detectable label.

33. The antibody or antigen-binding fragment of claim 32, wherein said at least a first label is a radiolabel, a chromogenic label, a fluorescent label or a labelled secondary antibody that specifically binds to said peptide, said polypeptide, or said antibody.

34. The antibody or antigen-binding fragment of claim 33, wherein said radiolabel comprises an ³H, a ¹⁴C, a ³²P, a ³⁵S, a ⁹⁰Y, a ⁹⁹Tc, an ¹²⁵I,or an ¹³¹I label.

35. The antibody or antigen-binding fragment of claim 33, wherein said chromogenic label comprises alkaline phosphatase, peroxidase, β-glucuronidase, β-D-glucosidase, β-D-galactosidase, urease, glucose oxidase/peroxidase, or galactose oxidase/peroxidase.

36. The antibody or antigen-binding fragment of claim 33, wherein said fluorescent label comprises a fluorescent protein, fluorescein, rhodamine, or auramine.

37. The antibody or antigen-binding fragment of claim 36, wherein said fluorescent protein comprises at least a first green fluorescent protein or at least a first humanized green fluorescent protein.

38. A polyclonal antiserum comprising a plurality of antibodies immunospecific for the peptide of claim 1, the polypeptide of claim 11, or a native, or biologically-active mammalian p33^{QIK} or p63^{Krsl} peptide or polypeptide.

39. A plurality of purified polyclonal antibodies obtained from the antiserum of claim 38, said plurality of purified polyclonal antibodies immunospecific for the peptide of claim 1, the polypeptide of claim 11, or a native, or biologically-active mammalian p33^{QIK} or p63^{Krsl} peptide or polypeptide.

40. A monoclonal antibody that specifically binds to a native, or biologically active mammalian p33^{QIK} or p63^{Krsl} peptide or polypeptide, but does not bind to a mammalian p63^{Krsl} peptide or polypeptide denatured under conditions of SDS-PAGE.

41. A hybridoma cell capable of producing at least a first antibody that is immunospecific for the peptide of claim 1, the polypeptide of claim 11, or a native, or biologically-active mammalian p33^{QIK} or p63^{Krsl} peptide or polypeptide.

42. A composition comprising:
(a) an isolated peptide of from 9 to about 70 amino acids in length, said peptide comprising at least a first contiguous amino acid sequence according to any one of SEQ ID NO:3 through SEQ ID NO:76;
(b) an isolated polypeptide consisting essentially of the amino acid sequence from position 1 to position 322 of SEQ ID NO:2;
(c) a native, or biologically-active mammalian p33^{QIK} or p63^{Krsl} peptide or polypeptide; or
(d) a purified antibody, polyclonal antiserum, or antigen-binding fragment thereof, that is immunospecific for the peptide of (a), the polypeptide of (b), or the peptide of polypeptide of (c), or a hybridoma cell capable of producing said antibody.

43. The composition of claim 42, further comprising at least a first pharmaceutically-acceptable excipient.

44. The composition of claim 42, further comprising at least a first immunostimulant or at least a first adjuvant.

45. The composition of claim 44, wherein said at least a first immunostimulant or said at least a first adjuvant is selected from the group consisting of a cytokine, a microsphere, Ribi Adjuvant, saponin, a microfluidized adjuvant, an immune stimulating complex, and an inactivated toxin.

46. The composition of claim 42, wherein said composition is formulated for parenteral, intravenous, intraperitoneal, subcutaneous, intranasal, transdermal, or oral administration to an animal.

47. The composition of claim 42, further comprising at least a first detection reagent.

48. The composition of claim 47, wherein said detection reagent comprises a radiolabel, a spin label, or a fluorogenic, chromogenic, or a chemiluminescent label.

49. The composition of claim 48, wherein said detection reagent specifically binds to a p33^{QIK} or p63^{Krsl} peptide or polypeptide, or to an antibody or an antigen binding fragment specific for a p33^{QIK} or p63^{Krsl} peptide or polypeptide.

50. A kit comprising:
(a) (i) an isolated peptide of from 9 to about 70 amino acids in length, said peptide comprising at least a first contiguous amino acid sequence according to any one of SEQ ID NO:3 through SEQ ID NO:76;
(ii) an isolated polypeptide consisting essentially of the amino acid sequence from position 1 to position 322 of SEQ ID NO:2;
(iii) a native, or biologically-active mammalian p33^{QIK} or p63^{Krsl} peptide or polypeptide; or
(iv) a purified antibody, polyclonal antiserum, or antigen-binding fragment thereof, that is immunospecific for the peptide of (i), the polypeptide of (ii), or the peptide of polypeptide of (iii), or a hybridoma cell capable of producing said antibody; and
(b) instructions for using said kit.

51. The kit of claim 50, wherein said kit comprises at least one component for performing immunoprecipitation, a dot blot, an ELISA, an RIA, or a Western blot.

52. The kit of claim 50, wherein said kit comprises at least one component for immunoprecipitating a native or biologically-active mammalian p33^{QIK} or p63^{Krsl} polypeptide from a sample.

53. A method of detecting a mammalian p33^{QIK} or p63^{Krsl} peptide, polypeptide, or protein in a sample comprising contacting a sample suspected of containing said peptide, polypeptide, or protein with the antibody of claim 13, the polyclonal antiserum of claim 38, the plurality of antibodies of claim 39, or the monoclonal antibody of claim 40, under conditions effective to produce immune complexes and detecting the complexes so formed.

54. The method of claim 53, wherein said sample is a biological sample.

55. The method of claim 54, wherein said sample is obtained from a human.

56. The method of claim 55, wherein said sample is a clinical sample obtained from a patient having, suspected of having, or at risk for developing, a p33^{QIK} or p63^{Krsl}-mediated disease, and further wherein an elevated amount of said peptide, polypeptide or protein in comparison to the amount present in a clinical sample obtained from a healthy human is indicative of said disease.

57. A method of generating an immune or a T-cell response in an animal comprising administering to said animal a composition that comprises at least a first isolated peptide according to claim 1, or at least a first isolated polypetpide according to claim 11, or at least a first nucleic acid segment that encodes said peptide or said polypeptide, in an amount and for a time sufficient to generate said immune or said T-cell response in said animal.

58. The method of claim 57, wherein said animal is a human.

59. A method of inhibiting the biological activity of a p33^{QIK} or p63^{Krsl} peptide or polypeptide in a cell, comprising providing to said cell at least a first composition that comprises: (a) the antibody of claim 13; (b) the polyclonal antiserum of claim 38; (c) the plurality of antibodies of claim 39; or (d) the monoclonal antibody of claim 40, in an amount effective and for a time sufficient to inhibit the biological activity of said peptide or polypeptide in said cell.

60. The method of claim 59, wherein said cell is comprised within an animal.
